(19) United States
(12) Patent Application Publication
(10) Pub. No.: US 2011/0160514 A1
(43) Pub. Date: Jun. 30, 2011

(54) ELECTRICAL ABLATION DEVICES

(75) Inventors: Gary L. Long, Cincinnati, OH (US); David N. Plescia, Cincinnati, OH (US); Peter K. Shires, Hamilton, OH (US)

(73) Assignee: Ethicon Endo-Surgery, Inc., Cincinnati, OH (US)

(21) Appl. No.: 12/651,181

(22) Filed: Dec. 31, 2009

(51) Int. Cl. A61B 18/02; A61B 18/14; A61N 5/00; A61B 17/22

(52) U.S. Cl. 600/2; 606/41; 606/21; 606/128

(57) ABSTRACT

A variety of electrical ablation apparatuses and methods are disclosed. In one embodiment, an ablation apparatus includes an injector catheter electrode having a proximal end configured to couple to an energy source and a fluid source. A distal end of the injector catheter defines an injection needle and defines an electrically conductive hollow channel for communicating a fluid from the fluid source to a treatment site. A balloon electrode is in fluid communication with a balloon catheter. The balloon catheter has a proximal end configured to couple to the energy source and the fluid source and a distal end configured to inflate the balloon electrode.

### BACKGROUND

Conventional ablation techniques such as thermal and chemical ablation therapy, among others, have been employed in medicine for the treatment of abnormal or undesirable tissue particularly diseased tissue including cancer, malignant and benign tumors, masses, lesions, and other abnormal growths. Thermal ablation techniques employ electrical ablation apparatuses, systems, and methods for treating tissue using electrically generated thermal energy. Although such electrical ablation techniques are generally effective for the treatment of abnormal tissue, electrically generated thermal ablation treatment is likely to cause permanent damage to healthy tissue surrounding the abnormal tissue under treatment. Permanent damage to healthy tissue is primarily due to exposure to detrimental thermal energy generated by the electrical ablation device. This is particularly true when tissue is exposed to electric potentials sufficient to cause cell necrosis. Most often this is a result of therapies that employ high temperature focused ultrasound ablation, radiofrequency (RF) ablation, interstitial laser coagulation, or similar high energy thermal ablation techniques. Another trend in tissue ablation therapy is injecting chemical agents into tissue to remove abnormal or undesirable tissue. Still other conventional ablation techniques include surgical excision, cryogenic therapy (cryotherapy), radiation, photodynamic therapy, Moh's micrographic surgery, topical treatments with 5-fluorouracil, laser ablation. Damage inflicted on healthy tissue caused by these conventional ablation therapies is compounded by high cost, long recovery periods, and extraordinary pain inflicted on the patient.

Conventional ablation techniques are employed in the treatment of a variety of undesirable tissues, although with less than optimal results, such as the treatment or removal of sessile polyps in the colon, liver tumors, hyperplastic cells in the prostrate gland, and liver malignancies such as hepatocellular cancer (HCC) and colorectal liver metastases (CRLM).

The removal or treatment of sessile polyps in the colon using conventional ablation techniques can be difficult because the polyps are hard to reach. Sessile polyps in particular are difficult to remove due to their low profile and thus are difficult to lasso with a snare in attempt to surgically remove them. Conventional techniques are prone to tearing the thin colon wall, which could have devastating effects on the patient.

Conventional ablation techniques have been used to treat liver tumors. These tumors are typically three to five centimeters in diameter and lay deep in the liver tissue. It is difficult to remove liver tumors using conventional ablation techniques because it is difficult to reach the tumors and the application of high energy thermal ablation can cause too much damage to the healthy liver tissue surrounding the tumor.

Thermal ablation techniques have been employed to ablate hyperplastic cells in the prostrate gland to reduce the size of the prostrate. This treatment is complicated by the location of the prostrate and the application of high energy thermal ablation can cause too much damage to the surrounding bladder or to the tissue interface between the prostrate and the rectum.

Hepatocellular cancer (HCC) and colorectal liver metastases (CRLM) are two of the most common hepatic malignancies treated with conventional ablation techniques. Although these liver malignancies are growing worldwide, HCC is more prevalent in Eastern countries due to cirrhosis and hepatitis, whereas CRLM occurs more commonly in Western countries such as the United States. The incidence of HCC, however, is growing worldwide. Patients with HCC are often not candidates for resection due to the underlying disease, whereas 75% of CRLM are not resectable at all. HCC begins in the hepatocytes as the result of liver damage (cirrhosis, hepatitis) and harvests its blood supply from the hepatic artery and becomes hypervascular. CRLM begins when cells from tumors in the colon travel through the portal vein and plant themselves anywhere in the liver. These hepatic malignancies form a blood supply in anyway they can and will grow rapidly, eventually becoming hypovascular in the center and hypervascular on the outside.

Due to the unique differences between HCC and CRLM, different instruments are employed to treat these malignancies. Conventional treatment alternatives for HCC and CRLM hepatic malignancies include percutaneous ethanol ablation (PEI), transcatheter embolization (TACE), and ablation. Ablation is performed as an open procedure, laparoscopically, or percutaneously. Due in part to the difficulty of accessing the liver in open or percutaneous procedures, the recurrence rate after ablation has been reported to be about 3.5% and 26.4% (p<0.0001) for open and percutaneous procedures respectively. Yet the rate of morbidity has been shown to be 15.3% and 2.4%, (p=0.044), for surgical and percutaneous procedures, respectively. The effectiveness of treatment and reduction in morbidity in the treatment of HCC and CRLM may be improved by employing Natural Orifice Translumenal Endoscopic Surgery (NOTES)™ techniques, developed by Ethicon Endosurgery, Inc., or a combination of NOTES™ and percutaneous procedures.

Although a variety of techniques have been developed for treating undesirable or abnormal tissue using thermal and non-thermal ablation systems, such techniques do not overcome the limitations set forth above. Accordingly, there remains a need for improved electrical ablation apparatuses, systems, and methods for the treatment of undesirable tissue found in diseased tissue, cancer, malignant and benign tumors, masses, lesions, and other abnormal tissue growths. There also remains a need for improved minimally invasive treatment of tissue through the use of irreversible electroporation (IRE) ablation techniques to minimize detrimental thermal effects to healthy tissue caused by conventional thermal ablation techniques.

### FIGURES

The novel features of the various embodiments disclosed in the specification are set forth with particularity in the appended claims. The various disclosed embodiments, however, both as to organization and methods of operation, together with advantages thereof, may be understood in accordance with the following description taken in conjunction with the accompanying drawings as follows.

FIG. 1 illustrates one embodiment of an electrical ablation system.

FIGS. 2-9 illustrate one embodiment of a sequence for removing or treating tissue in a tissue treatment region using a pulsed direct current (DC) electroporation tissue ablation treatment technique.

FIG. 2 illustrates an endoscope partially introduced into the colon of a patient for the treatment of a polyp growing on a wall of the colon.

FIG. 3 illustrates the wall of the colon in cross-section and the endoscope partially introduced in the colon.

FIG. 4 illustrates one embodiment of an injector catheter electrode introduced into the treatment region proximate to the polyp shown in FIGS. 2 and 3.

FIG. 5 illustrates the injector catheter electrode shown in FIG. 4 inserted into the polyp where saline is injected to form a bleb to lift or raise the polyp away from the interior portion of the wall of the colon.

FIG. 6 illustrates one embodiment of a balloon catheter introduced into the treatment region proximate the polyp.

FIG. 7 illustrates one embodiment of a conductive elastomer portion of a balloon electrode acting as a second electrode to conduct electricity from an injection needle electrode through the bleb, the polyp, the balloon electrode, and back to the energy source.

FIG. 8 illustrates a change in the bleb and the polyp shown in FIGS. 5-7 after the irreversible electroporation treatment is applied.

FIG. 9 is a graphical representation of electric field strength in volts per meter (V/m) developed across the polyp shown in FIGS. 5-8 when the injection needle is energized by the energy source shown in FIG. 1 and the balloon electrode acts as a return.

FIGS. 10-12 illustrate one embodiment of an ablation device for treating tumors embedded in a larger mass of tissue.

FIG. 10 is a cross-sectional sectional view of a liver showing a tumor embedded in a single lobe of the liver and one embodiment of an ablation device piercing through the tumor and clamping the single lobe.

FIG. 10A illustrates one embodiment of the ablation device shown in FIG. 10.

FIGS. 11 and 12 are graphical representations of the electric field applied to the treatment region showing where necrosis will occur in the ablation zone around the tumor when the third electrode is energized with a pulsed positive potential and the first and second electrodes are connected to ground.

FIG. 11 illustrates an end view of the ablation zone.

FIG. 12 illustrates a side view of the ablation zone.

FIGS. 13 and 14 illustrate implementations of thermal ablation techniques for ablating hyperplastic cells in the prostrate gland to reduce the size of the prostrate.

FIG. 13 is a cross-sectional view of the male pelvis and one embodiment of an electrical ablation system for ablation treatment of the prostate by applying high voltage direct current (DC) pulses in the treatment region.

FIG. 14 is finite element model of the electric field created in the prostrate when the electrodes shown in FIG. 13 are energized.

FIGS. 15 and 16 illustrate a hepatic tumor before and after treatment by the application of high voltage direct current (DC) pulses with an ablation system.

FIG. 15 is a radiological image illustrating a first electrode placed in the tumor and a separate second electrode placed intravenously through the hepatic artery.

FIG. 16 is a radiological image illustrating the ablation zone that is outlined by obliterated capillaries from the high voltage direct current (DC) pulse treatment.

FIG. 17 shows a liver, a tumor, and one embodiment of a probe placed into the tumor.

FIG. 18 is a detailed view of the probe and the tumor.

FIG. 19 is a graphical representation of electric field strength in two discernible zones of electric field strength created at distances proximate to the center of an energized electrode.

FIGS. 20A-E illustrate one implementation of a method of debulking a tumor and causing a specific systemic response by employing electroporation techniques.

FIG. 20A shows a tumor embedded inside the liver.

FIG. 20B shows a catheter inserted into the liver and advanced to a position proximate to the tumor to treat the tumor.

FIG. 20C shows a distal end of a central electrode inserted into the tumor and a plurality of injection needles surrounding the tumor.

FIG. 20D shows the formation of a necrotic zone created by a combination of electrical pulses delivered by the central electrode shown in FIG. 20C and the injection of DNA plasmid with the injection needles shown in FIG. 20C.

FIG. 20E shows the formation of a reversible zone created by energizing the central electrode shown in FIG. 20C.

FIG. 21 is a graphical illustration of a pulse train composed of a plurality of pulse bursts produced by the energy source shown in FIG. 1 which can be applied to the central electrode shown in FIG. 20C for the treatment or debulking of a tumor, where each pulse burst is composed of a plurality of pulses.

FIG. 21A is a detail view of two of the individual pulses that compose each of the pulse bursts shown in FIG. 21.

FIGS. 22-24 illustrate one embodiment of an ablation device comprising an inner electrode and an outer electrode which are non-parallel to each other.

FIG. 22 shows the inner and outer electrodes proximate to the liver.

FIG. 23 shows the inner and outer electrodes embedded in the liver.

FIG. 24 shows the inner and outer electrodes embedded in the liver from a different angle than shown in FIG. 23.

FIG. 25 shows the outer electrode shown in FIG. 22 advanced into the tumor while a finite element solution of a threshold of necrosis showing the electric field isosurface defining the boundary of the necrotic zone is constantly updated and overlaid on a computed tomography (CT) image.

FIG. 26 is a diagram illustrating a combined cryogenic and irreversible electroporation (IRE) treatment of a tumor.

FIG. 27 is a graphic representation of necrotic threshold plot where electric field threshold of cell death is plotted versus pulse width.

FIG. 28 illustrates one embodiment of a probe comprising electrodes, which can deliver unipolar pulses of about 250 ns to a tissue treatment site to cause necrotic cell death.

FIG. 29 shows the un-insulated conductive portions of two electrodes fully embedded into tissue and tumor.

FIG. 30 shows the un-insulated conductive portions of two electrodes not fully embedded into tissue and tumor leaving an air gap for creating an arc.

FIG. 31 illustrates one embodiment of a device for producing an acoustic wave using high voltage discharge.

FIG. 32 shows the device shown in FIG. 32 with the dome-like structure in contact with a stone.

### DESCRIPTION

The various embodiments disclosed in the present specification are directed generally to apparatuses, systems, and methods for electrical ablation treatment of undesirable tissue such as diseased tissue, cancer, malignant and benign tumors, masses, lesions, and other abnormal tissue growths while minimizing or eliminating detrimental effects to surrounding healthy tissue. Numerous specific details are set forth to provide a thorough understanding of the overall structure, function, manufacture, and use of the disclosed embodiments as described in the specification and illustrated in the accompanying drawings. It will be understood by those skilled in the art, however, that the disclosed embodiments may be practiced without the specific details disclosed herein. In other instances, well-known operations, components, and elements have not been described in detail in the interest of conciseness and clarity and so as not to obscure the disclosed embodiments. Those of ordinary skill in the art will understand that the disclosed embodiments serve as non-limiting examples and thus it can be appreciated that the specific structural and functional details disclosed herein are representative in nature and are not necessarily limiting. Rather, the overall scope of the embodiments is defined solely by the appended claims.

Reference throughout the specification to “various embodiments,” “some embodiments,” “one embodiment,” or “an embodiment” means that a particular feature, structure, or functional characteristic described in connection with a disclosed embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the particular features, structures, or functional characteristics of one or more than one embodiments may be combined in any suitable manner, without limitation. Thus, the particular features, structures, or functional characteristics illustrated or described in connection with one embodiment may be combined, in whole or in part, with the features structures, or characteristics of one or more than one other embodiment without limitation.

It will be appreciated that the terms “proximal” and “distal” may be used throughout the specification with reference to a clinician manipulating one end of an instrument used to treat a patient. In this context, the term “proximal” refers to the portion of the instrument located closest to the clinician and the term “distal” refers to the portion located furthest from the clinician. It will be further appreciated that for the sake of conciseness and clarity, spatial terms such as “vertical,” “horizontal,” “up,” and “down” may be used herein with respect to a disclosed embodiment. However, surgical instruments may be used in many orientations and positions, and these terms are not intended to be limiting and absolute.

Various embodiments of apparatuses, systems, and methods for the electrical ablation treatment of undesirable tissue such as diseased tissue, cancer, malignant and benign tumors, masses, lesions, and other abnormal tissue growths, are described throughout the specification and illustrated in the accompanying drawings. The electrical ablation devices in accordance with the disclosed embodiments may comprise one or more than one electrode configured to be positioned into or proximal to undesirable tissue within a tissue treatment region (e.g., target site, worksite) where there is evidence of abnormal tissue growth, for example. In general, the electrodes comprise an electrically conductive portion (e.g., medical grade stainless steel among other suitable biologically compatible conductive materials) and are configured to electrically couple to an energy source. Once the electrodes are positioned into or proximal to the undesirable tissue, an energizing potential is applied to the electrodes thus exposing the undesirable tissue to an electric field. The energizing potential (and the resulting electric field) may be characterized by multiple parameters such as frequency, amplitude, pulse width (duration of a pulse or pulse length), and/or polarity. Depending on the diagnostic or therapeutic treatment to be rendered, a particular electrode may be configured either as an anode (+) or a cathode (−) or may comprise a plurality of electrodes with at least one configured as an anode and at least one other configured as a cathode. Regardless of the initial polar configuration, the polarity of the electrodes may be reversed by reversing the polarity of the output of the energy source.

In various embodiments, a suitable energy source may comprise an electrical waveform generator, which may be configured to create electric fields suitable for creating irreversible electroporation in undesirable tissue at various electric field amplitudes, frequencies, and/or and durations. The energy source may be configured to deliver irreversible electroporation pulses in the form of direct-current (DC) and/or alternating-current (AC) voltage potentials (e.g., time-varying voltage potentials) to the electrodes (as well as the potential reversing between the electrodes). The irreversible electroporation pulses may be characterized by various parameters such as frequency, amplitude, pulse length, and/or polarity. The undesirable tissue may be ablated by exposure to the electric potential difference across the electrodes.

Unipolar as well as bipolar pulses have been shown to cause cell necrosis by immediately destroying the cell plasma membrane as well as triggering cell apoptosis. There are advantages to both of these cellular death mechanisms. Although causing immediate cell death is preferred if the cells are malignant, cell apoptosis is a more natural form of cellular death and is therefore more compatible with the way in which the immune system “cleans up” dead cells. In one embodiment, a method and a device is disclosed which causes cell death with unipolar pulses which are nominally about 250 nanoseconds in pulse duration.

High voltage DC pulses of several thousand volts ranging in duration from a few nanoseconds to a few tens of microseconds may be employed to cause cell necrosis in-vivo. During the application of such a pulse, an acoustic wave (usually audible), likely due to the rapid change in voltage at the electrode surface, is generated, which can be used to break stones in the kidney.

In one embodiment, the energy source may comprise a wireless transmitter to deliver energy to the electrodes using wireless energy transfer techniques via one or more remotely positioned antennas or inductive coils. Those skilled in the art will appreciate that wireless energy transfer or wireless power transmission is the process of transmitting electrical energy from an energy source to an electrical load without interconnecting wires. An electrical transformer is the simplest instance of wireless energy transfer. The primary and secondary circuits of a transformer are not directly connected and the transfer of energy takes place by electromagnetic coupling through a process known as mutual induction. Power also may be transferred wirelessly using RF energy.

In various embodiments, the system can remotely power energy consuming modules located within a patient, for example, using wireless energy transfer techniques such as inductive coupling, resonant, or RF wireless energy transfer techniques. Such wireless energy coupling techniques use AC magnetic fields generated in a first inductive coil (e.g., conductor) located outside the patient to stimulate electrical current through a second inductive coil (e.g., conductor) located inside the patient. Wireless energy transfer or wireless power transmission is the process of transmitting electrical energy from an energy source to an electronic load, without interconnecting wires, using electromagnetic fields. An electronic transformer is the simplest instance of wireless energy transfer. The primary and secondary circuits of a transformer are not directly connected. The transfer of energy takes place by electromagnetic coupling through a process known as mutual induction. Wireless power transfer technology using RF energy produced by Powercast, Inc. also may be employed without limitation. For example, the Powercast system achieves a maximum output of 6 volts for a little over one meter. Other low-power wireless power technology has been proposed and is described in U.S. Pat. No. 6,967,462, for example.

In one embodiment, a wireless energy transmitter module is coupled to an energy source, which provides a suitable power (voltage and current) to a wireless energy transmitter module. A generator circuit converts the power received from the energy source and supplies AC power to a generating element. In various embodiments, the generating element may comprise one or more than one single or multi-turn inductive coil, for example. In one embodiment, an energy consuming module comprises a wireless energy module, which comprises a collection element to receive energy generated by the generating element. In one embodiment, the collection element may comprise one or more than one single or multi-turn inductive coil, for example. The transfer of energy from the generating element to the collection element may be via inductive coupling, or via resonant energy transfer, for example, in both instances without employing wires. Thus, energy is transmitted wirelessly via inductive coupling from the manipulation unit to the energy consuming module, e.g., across the abdominal wall of a patient.

The collection element of the wireless energy module is coupled to a conditioning circuit that generates a suitable operating voltage and current for use by an electronic component. In one embodiment, the conditioning circuit may be coupled to an optional rechargeable battery that can be charged using the energy transferred to the collection element. The battery is recharged by the combination of the generating element (e.g., generating coil) and the collection element (e.g., collection coil) with the conditioning circuit providing voltage and current outputs suitable for recharging the rechargeable battery. Alternatively, a capacitor may be charged to store energy and power attached circuits. Inductive coupling uses magnetic fields that are generated by the movement of electric current through the wire forming the generating element. The magnetic field induces a current in the collection element. As is well known in the art, when electrical current moves through a wire, it creates a circular magnetic field around the wire. Bending the wire into a first coil amplifies the magnetic field. The more loops the coil makes, the bigger the field will be. If a second coil of wire is placed in the magnetic field, the field can induce a current in the wire of the second coil. This is essentially how a transformer works and how the wireless energy module supplies energy to an electronic component and/or recharges the battery or capacitive circuit by inductive coupling. Current from the energy source flows through the generator circuit and the generating element (e.g., first coil) portion of the wireless energy transmitter module, creating a magnetic field. In a transformer, the first coil is called the primary winding. When the wireless energy transmitter module is energized and placed near the wireless energy module, the magnetic field generated by the first coil induces a current in the energy collection element (e.g., second coil), or secondary winding, which connects to the conditioning circuit and/or the battery. The conditioning circuit converts this current into a suitable voltage and current for operating the electronic component by or for recharging the battery.

The apparatuses, systems, and methods in accordance with the disclosed embodiments may be configured for minimally invasive ablation treatment of undesirable tissue through the use of irreversible electroporation in order to ablate undesirable tissue in a controlled and focused manner without inducing damaging thermal effects to healthy tissue surrounding the undesirable tissue under treatment. The apparatuses, systems, and methods in accordance with the disclosed embodiments may be configured for ablating undesirable tissue through the use of electroporation or electropermeabilization. More specifically, the apparatuses, systems, and methods in accordance with the disclosed embodiments may be configured for ablating undesirable tissue through the use of irreversible electroporation. Electroporation increases the permeabilization of a cell membrane by exposing the cell to electric pulses. The external electric field (electric potential/per unit length) to which the cell membrane is exposed to significantly increases the electrical conductivity and permeability of the plasma in the cell membrane. The primary parameter affecting the transmembrane potential is the potential difference across the cell membrane. Irreversible electroporation is the application of an electric field of a specific magnitude and duration to a cell membrane such that the permeabilization of the cell membrane cannot be reversed, leading to cell death but without inducing a significant amount of heat in the cell membrane. The destabilizing potential forms pores in the cell membrane when the potential across the cell membrane exceeds its dielectric strength causing the cell to die under a process known as apoptosis and/or necrosis. The application of irreversible electroporation pulses to the cellular structure is an effective way of ablating large volumes of undesirable tissue without deleterious thermal effects to the healthy tissue surrounding the undesirable tissue under treatment, which is associated with thermal-inducing ablation treatments. This is because irreversible electroporation destroys cells without heat and thus does not destroy the cellular support structure or regional vasculature. A destabilizing irreversible electroporation pulse, suitable for causing cell death without inducing a significant amount of thermal damage to the surrounding healthy tissue, may have amplitude in the range of about several hundred to about several thousand volts and is generally applied across biological membranes over a distance of about several millimeters and may be applied for a duration from about a few nanoseconds to about a few seconds, for example. Thus, the undesirable tissue may be ablated in-vivo through the delivery of destabilizing electric fields by quickly creating cell necrosis.

The apparatuses, systems, and methods for electrical ablation therapy in accordance with the disclosed embodiments may be adapted for use in minimally invasive surgical procedures to access the tissue treatment region in various anatomic locations such as the brain, lungs, breast, liver, gall bladder, pancreas, prostate gland, and various internal body lumen defined by the esophagus, stomach, intestine, colon, arteries, veins, anus, vagina, cervix, fallopian tubes, and/or the peritoneal cavity, for example, without limitation. Minimally invasive electrical ablation devices may be introduced to the tissue treatment region using a trocar inserted though a small opening formed in the patient's body or through a natural body orifice such as the mouth, anus, or vagina using translumenal access techniques known as NOTES™. Once the electrical ablation devices (e.g., electrodes) are located into or proximal to the undesirable tissue in the treatment region, electric field potentials can be applied to the undesirable tissue by the energy source. The electrical ablation devices comprise portions that may be inserted into the tissue treatment region percutaneously (e.g., where access to inner organs or other tissue is done via needle-puncture of the skin). Other portions of the electrical ablation devices may be introduced into the tissue treatment region endoscopically (e.g., laparoscopically and/or thoracoscopically) through trocars or channels of the endoscope, through small incisions, or transcutaneously (e.g., where electric pulses are delivered to the tissue treatment region through the skin).

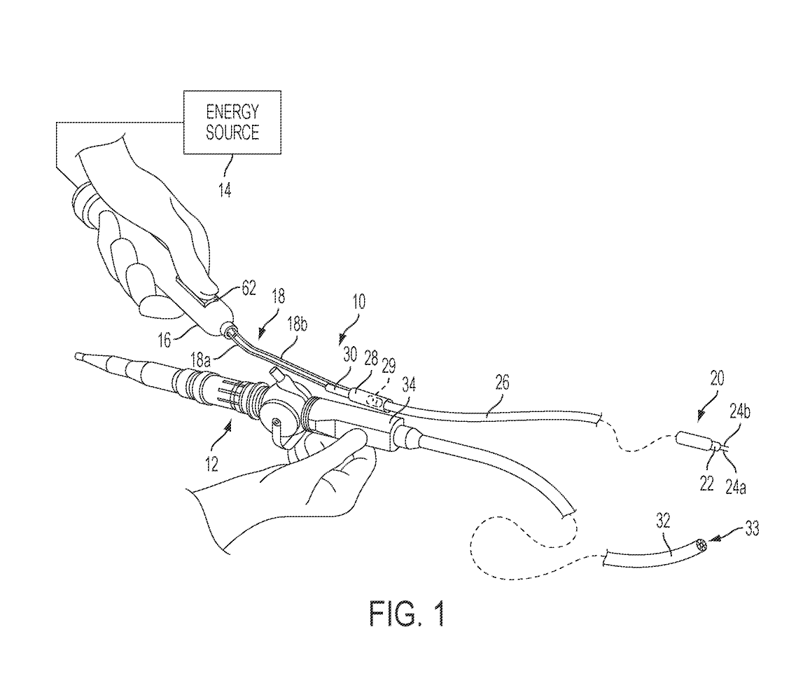

With this general background description of the disclosed embodiment, the description now turns to FIG. 1, which illustrates one embodiment of an electrical ablation system 10. The electrical ablation system 10 may be employed to ablate undesirable tissue such as diseased tissues, cancers, tumors, masses, lesions, abnormal tissue growths inside a patient using electrical energy. The electrical ablation system 10 may be used in conjunction with endoscopic, laparoscopic, thoracoscopic, open surgical procedures via small incisions, keyholes, percutaneous techniques, transcutaneous techniques, and/or external non-invasive techniques, or any combinations thereof without limitation. The electrical ablation system 10 may be configured to be positioned within a natural body orifice of the patient such as the mouth, anus, or vagina and advanced through internal body lumen or cavities such as the esophagus, colon, cervix, uterus, urethra, bladder, for example, to reach the tissue treatment region. The electrical ablation system 10 also may be configured to be positioned and passed through a small incision or keyhole formed through the skin and/or abdominal wall of the patient to reach the tissue treatment region using a trocar. The tissue treatment region may be located anywhere within the patient such as the brain, lungs, breast, liver, gall bladder, pancreas, kidneys, prostate gland, various internal body lumen defined by the esophagus, stomach, intestine, colon, arteries, veins, anus, vagina, cervix, uterus, fallopian tubes, urethra, bladder, and/or the peritoneal cavity, for example, without limitation. The electrical ablation system 10 can be configured to treat a number of lesions and ostepathologies comprising metastatic lesions, tumors, fractures, infected sites, inflamed sites.

Once positioned into or proximate the tissue treatment region, the electrical ablation system 10 can be actuated (e.g., energized) to ablate the undesirable tissue. In one embodiment, the electrical ablation system 10 may be configured to treat undesirable tissue in the gastrointestinal (GI) tract, esophagus, lung, or stomach that may be accessed orally. In another embodiment, the electrical ablation system 10 may be adapted to treat undesirable tissue in the liver, gull bladder, kidneys or other organs that may be accessible using translumenal access techniques such as, without limitation, NOTES™ techniques, where the electrical ablation devices may be initially introduced through a natural orifice such as the mouth, anus, or vagina and then advanced to the tissue treatment site by puncturing the walls of internal body lumen such as the stomach, intestines, colon, cervix, uterus. In various embodiments, the electrical ablation system 10 may be adapted to treat undesirable tissue in the brain, lungs, breast, liver, gall bladder, pancreas, kidneys, bladder, or prostate gland, using one or more electrodes positioned percutaneously, transcutaneously, translumenally, minimally invasively, and/or through open surgical techniques, or any combination thereof.

In one embodiment, the electrical ablation system 10 may be employed in practice in conjunction with a flexible endoscope 12, as well as a rigid endoscope, laparoscope, or thoracoscope, such as the GIF-100 model available from Olympus Corporation. In one embodiment, the endoscope 12 may be introduced to the tissue treatment region trans-anally through the colon, trans-orally through the esophagus and stomach, trans-vaginally through the cervix or uterus, transcutaneously, or via an external incision or keyhole formed in the abdomen in conjunction with a trocar. The electrical ablation system 10 may be introduced into the tissue treatment region separately but in conjunction with the endoscope 12, as shown, such that the ablation system 10 and the endoscope 12 are introduced into the tissue treatment region at the same time. In other embodiments, the electrical ablation system 10, or elements thereof, may introduced and guided into or proximate the tissue treatment region through various channels formed within the endoscope 12.

In the embodiment illustrated in FIG. 1, the endoscope 12 comprises an endoscope handle 34 and an elongate relatively flexible shaft 32. The distal end 33 of the flexible shaft 32 may comprise an air/water nozzle, a light guide, a viewing port comprising an objective lens, various instrument channels, and various auxiliary channels. A light source is provided to illuminate the area to be viewed through the viewing port. Optionally, one or more than one of the various instrument channels or the auxiliary channels defined within the flexible shaft 32 are suitable for receiving various instruments therethrough, such as electrical ablation system 10 or elements thereof, for example. Images within the field of view of the viewing port are received by an optical device, such as a camera comprising a charge coupled device (CCD) or complementary metal-oxide semiconductor (CMOS) based image sensor usually located within the endoscope 12, and are transmitted to a display monitor (not shown) outside the patient. In other embodiments, fiber optic channels may be employed to carry the light signals to the optical device.

In one embodiment, the electrical ablation system 10 may comprise an electrical ablation device 20, a plurality of electrical conductors 18, a handpiece 16 comprising an activation switch 62, and an energy source 14 such as an electrical waveform generator, electrically coupled to the activation switch 62 and the electrical ablation device 20. Although in the illustrated embodiment the activation switch 62 is shown as part of the handpiece 16, it will be appreciated that the activation switch 62 may be integrated into a variety of other activation mechanisms such as a foot activated switch, without limitation. In one embodiment, the electrical ablation device 20 comprises a relatively flexible member or shaft 22 that may be introduced to the tissue treatment region using a variety of known techniques such as an open incision and a trocar, percutaneously, transcutaneously, or through one of more than one channel of the endoscope 12.

In one embodiment, one or more than one electrode such as first and second electrodes 24a,b extend out from the distal end of the electrical ablation device 20. In one embodiment, the first electrode 24a may be configured as the positive electrode and the second electrode 24b may be configured as the negative electrode. The first electrode 24a is electrically connected to a first electrical conductor 18a, or similar electrically conductive lead or wire, which is coupled to the positive terminal of the energy source 14 through the activation switch 62. The second electrode 24b is electrically connected to a second electrical conductor 18b, or similar electrically conductive lead or wire, which is coupled to the negative terminal of the energy source 14 through the activation switch 62. The electrical conductors 18a,b are electrically insulated from each other and surrounding structures, except for the electrical connections to the respective electrodes 24a,b. In various embodiments, the electrical ablation device 20 may be configured to be introduced into or proximate the tissue treatment region using the endoscope 12 (laparoscope or thoracoscope), open surgical procedures, or external and non-invasive medical procedures. The electrodes 24a,b may be referred to herein as endoscopic or laparoscopic electrodes, although variations thereof may be inserted transcutaneously or percutaneously. As previously discussed, either one or both electrodes 24a,b may be adapted and configured to slidably move in and out of a cannula, lumen, catheter, one or more than one channel defined within the flexible shaft 22, and/or one or more than one channel defined within the flexible shaft 32 of the endoscope 12. In various embodiments, the electrodes 24a, 24b may be configured in a variety of structures and forms. For example, such electrodes may be configured as needles, balloons that can be inflated within internal body lumen, flat, tapered or chisel-shaped, cylindrical, rectangular, fixed, longitudinally or radially rotatable, slidable, extendable, retractable, or any combination thereof, without limitation. Furthermore, although two electrodes 24a, 24b are shown, each electrode 24a,b may comprise one or more than one electrode having any suitable configuration, such as, for example, the configurations disclosed within this specification, among other configurations.

Once the electrodes 24a,b are positioned at the desired location into or proximate the tissue treatment region, the electrodes 24a,b may be electrically connected to or disconnected from the energy source 14 by actuating or de-actuating the switch 62 on the handpiece 16. The switch 62 may be operated manually or may be mounted on a foot switch (not shown), for example. The switch 62 may be operated automatically as well as by the user. In automatic operation, for example, the switch 62 may be activated or deactivated in response to various measurable quantities such as electrical data (e.g., impedance, energy delivered, frequency, amplitude, pulse width), imaging data (e.g., optical recognition of the tissue treatment region derived from the image sensor), acoustic data (e.g., ultrasonic imaging signals of the tissue treatment region), without limitation. It will be appreciated that in automatic mode, the electrical ablation system 10 may comprise additional analog and/or digital processing circuits, including processors such as digital signal processors or general purpose processors.

In one embodiment, the electrodes 24a,b are configured to deliver pulsed electric fields to the undesirable tissue. The electric field pulses may be characterized in terms of various parameters such as pulse shape, amplitude, frequency, and duration. The electric field pulses may be sufficient to induce irreversible electroporation in the undesirable tissue. The induced potential depends on a variety of conditions such as tissue type, cell size, and electrical pulse parameters. The primary electrical pulse parameter affecting the transmembrane potential for a specific tissue type is the amplitude of the electric field followed by the duration of the pulse that the tissue is exposed to.

In one embodiment, a protective sleeve or sheath 26 may be slidably disposed over the flexible shaft 22 and within a handle 28. In another embodiment, the sheath 26 may be slidably disposed within the flexible shaft 22 and the handle 28, without limitation. The sheath 26 is slidable and may be located over the electrodes 24a,b to protect the trocar and prevent accidental piercing when the electrical ablation device 20 is advanced therethrough. Either one or both of the electrodes 24a,b of the electrical ablation device 20 may be adapted and configured to slidably move in and out of a cannula, lumen, catheter, or channel formed within the flexible shaft 22. One of the electrodes, e.g., the second electrode 24b, may be fixed in place. The second electrode 24b may provide a pivot about which the first electrode 24a can be moved in an arc to other points in the tissue treatment region to treat larger portions of the diseased tissue that cannot be treated by fixing the electrodes 24a,b in one location. In one embodiment, either one or both of the electrodes 24a,b may be adapted and configured to slidably move in and out of one or more than one channel formed within the flexible shaft 32 of the flexible endoscope 12 or, as shown in FIG. 1, may be located independently of the flexible endoscope 12.

In one embodiment, the first and second electrical conductors 18a,b may be provided through the handle 28. In the illustrated embodiment, the first electrode 24a can be slidably moved in and out of the distal end of the flexible shaft 22 using a slide member 30 to retract and/or advance the first electrode 24a. In various embodiments either or both electrodes 24a,b may be coupled to the slide member 30, or additional slide members, to advance and retract all of the electrodes 24a,b, e.g., position the electrodes 24a,b. In the illustrated embodiment, the first electrical conductor 18a coupled to the first electrode 24a is coupled to the slide member 30. In this manner, the first electrode 24a, which is slidably movable within the cannula, lumen, catheter, or channel defined by the flexible shaft 22, can be advanced and retracted with the slide member 30.

In various other embodiments, transducers or sensors 29 may be located in the handle 28 of the electrical ablation device 20 to sense the force with which the electrodes 24a,b penetrate the tissue in the tissue treatment region. This feedback information may be useful to determine whether one or all of the electrodes 24a,b have been properly inserted in the tissue treatment region. As is particularly well known, cancerous tumor tissue tends to be denser than healthy tissue and thus greater force is required to insert the electrodes 24a,b therein. The transducers or sensors 29 can provide feedback to the operator, surgeon, or clinician to physically sense when the electrodes 24a,b are placed within the cancerous tumor. The feedback information provided by the transducers or sensors 29 may be detected, processed, and/or displayed by analog or digital circuits located either internally or externally to the energy source 14. The sensor 29 readings may be employed to determine whether the electrodes 24a,b have been properly located within the cancerous tumor thereby assuring that a suitable margin of error has been achieved in locating the electrodes 24a,b.

In one embodiment, the input to the energy source 14 may be connected to a commercial power supply (e.g., mains power such as the general-purpose AC power supply) by way of a plug (not shown). The output of the energy source 14 is coupled to the electrodes 24a,b, which may be energized using the activation switch 62 on the handpiece 16, an activation switch mounted on a foot activated pedal (not shown), and/or automatically based on feedback information received from electrical sensors (e.g., impedance, image sensors, acoustic transducers). The energy source 14 may be configured to produce electrical energy suitable for electrical ablation, as described in more detail below.

In one embodiment, the electrodes 24a,b are adapted and configured to electrically couple to the energy source 14 (e.g., generator, waveform generator). Once electrical energy is coupled to the electrodes 24a,b, an electric field is generated at a distal end of the electrodes 24a,b. The energy source 14 may be configured to generate static as well as pulsed electric fields at a predetermined frequency, amplitude, pulse length, and/or polarity that are suitable to induce irreversible electroporation in the cellular structure of the undesirable tissue for ablating substantial volumes of the undesirable tissue at the treatment region. For example, the energy source 14 may be configured to deliver DC electric pulses having a predetermined frequency, amplitude, pulse length, and/or polarity suitable to induce irreversible electroporation in cellular structure of the undesirable tissue for ablating substantial volumes of the undesirable tissue at the treatment region. The DC pulses may have a positive or negative polarity relative to a particular reference polarity. The polarity of the DC pulses may be reversed or inverted from positive-to-negative or negative-to-positive a predetermined number of times to induce irreversible electroporation to ablate substantial volumes of undesirable tissue at the treatment region.

In one embodiment, a timing circuit may be coupled to the output of the energy source 14 to generate electric pulses. The timing circuit may comprise one or more suitable switching elements to produce the electric pulses. For example, the energy source 14 may produce a series of n electric pulses (where n is any positive integer) of sufficient amplitude and duration to induce irreversible electroporation suitable for tissue ablation when the n electric pulses are applied to the electrodes 24a,b. In one embodiment, the electric pulses may have a fixed or variable pulse length, amplitude, and/or frequency.

In the illustrated embodiment, the energy source 14 may be configured to operate in either the bipolar or monopolar modes with the electrical ablation system 10. Accordingly, the electrical ablation device 20 may be configured to operate either in bipolar or monopolar mode. In bipolar mode, one of the electrodes 24a is electrically connected to a first polarity and another electrode 24b is electrically connected to the opposite polarity. When more than two electrodes are used, the polarity of the electrodes may be alternated such that any two adjacent electrodes may have either the same or opposite polarities, for example, or such that one electrode is coupled a particular polarity while the rest of the electrodes are coupled to the opposite polarity, without limitation.

In monopolar mode, the first electrode 24a is coupled to a prescribed voltage potential and the second electrode 24b is coupled to ground potential. In monopolar mode, it is not necessary that the patient be grounded with a grounding pad. Since a monopolar energy source 14 is typically constructed to operate upon sensing a ground pad connection to the patient, the negative electrode of the energy source 14 may be coupled to an impedance simulation circuit. In this manner, the impedance circuit simulates a connection to the ground pad and thus is able to activate the energy source 14. It will be appreciated that in monopolar mode, the impedance circuit can be electrically connected in series with either one of the electrodes 24a,b that would otherwise be attached to a grounding pad.

In one embodiment, the energy source 14 may be configured to produce RF waveforms at predetermined frequencies, amplitudes, pulse widths or durations, and/or polarities suitable for electrical ablation of cells in the tissue treatment region. One example of a suitable RF energy source is a commercially available conventional, bipolar/monopolar electrosurgical RF generator such as Model Number ICC 350, available from Erbe, GmbH.

In one embodiment, the energy source 14 may be configured to produce destabilizing electrical potentials (e.g., fields) suitable to induce irreversible electroporation. The destabilizing electrical potentials may be in the form of pulsed bipolar/monopolar DC electricity suitable for inducing irreversible electroporation to ablate tissue undesirable tissue with the electrical ablation device 20. A commercially available energy source suitable for generating irreversible electroporation electric filed pulses in bipolar or monopolar mode is a pulsed DC generator such as Model Number ECM 830, available from BTX Molecular Delivery Systems Boston, Mass. In bipolar mode, the first electrode 24a may be electrically coupled to a first polarity and the second electrode 24b may be electrically coupled to a second (e.g., opposite) polarity of the energy source 14. Bipolar/monopolar DC electric pulses may be produced at a variety of frequencies, amplitudes, pulse lengths, and/or polarities. Unlike RF ablation systems, however, which require high power and energy levels delivered into the tissue to heat and thermally destroy the tissue, irreversible electroporation requires very little energy input into the tissue to kill the undesirable tissue without the detrimental thermal effects because with irreversible electroporation the cells are destroyed by electric field potentials rather than heat.

In one embodiment, the energy source 14 may be coupled to the first and second electrodes 24a,b by either a wired or a wireless connection. In a wired connection, the energy source 14 is connected to the electrodes 24a,b by way of the electrical conductors 18a,b, as shown.

In a wireless connection, the electrical conductors 18a,b may be replaced with a first antenna or inductive coil (not shown) coupled the energy source 14 and a second antenna or inductive coil (not shown) coupled to the electrodes 24a,b, wherein the second antenna is remotely located from the first antenna. Accordingly, the energy source 14 may comprise a wireless transmitter to deliver energy to the electrodes using the previously described wireless energy transfer techniques. As previously discussed, wireless energy transfer or wireless power transmission is the process of transmitting electrical energy from the energy source 14 to an electrical load, e.g., the abnormal cells in the tissue treatment region, without using the interconnecting electrical conductors 18a,b.

In one embodiment, the energy source 14 may be configured to produce DC electric pulses at frequencies in the range of about 1 Hz to about 10,000 Hz, amplitudes in the range of about ±100 to about ±10,000VDC, and pulse lengths (e.g., pulse width, pulse duration) in the range of about 1 μs to about 100 ms. The polarity of the electric potentials coupled to the electrodes 24a,b may be reversed during the electrical ablation therapy. For example, initially, the DC electric pulses may have a positive polarity and an amplitude in the range of about +100 to about +10,000VDC. Subsequently, the polarity of the DC electric pulses may be reversed such that the amplitude is in the range of about −100 to about −10,000VDC. In one embodiment, the undesirable cells in the tissue treatment region may be electrically ablated with DC pulses suitable to induce irreversible electroporation at frequencies of about 10 Hz to about 100 Hz, amplitudes in the range of about +700 to about +1,500VDC, and pulse lengths of about 10 μs to about 50 μs. In another embodiment, the abnormal cells in the tissue treatment region may be electrically ablated with an electrical waveform having an amplitude of about +500VDC and pulse duration of about 20 ms delivered at a pulse period T or repetition rate, frequency f=1/T, of about 10 Hz.

In various embodiments, the energy source 14 is capable of generating electric fields with a strength ranging from a few hundred volts-per-centimeter (V/cm) to several tens-of-thousands of V/cm. For example, in various embodiments, the energy source can generate an electric field having a strength ranging from about 500V/cm to about 50,000V/cm, for example. It has been determined that an electric field having a strength of about 1,000V/cm is suitable for destroying living tissue by inducing irreversible electroporation. Treatment is performed by applying a sequence of pulses to the treatment site. The sequence of pulses may have any suitable amplitude, pulse duration, and frequency.

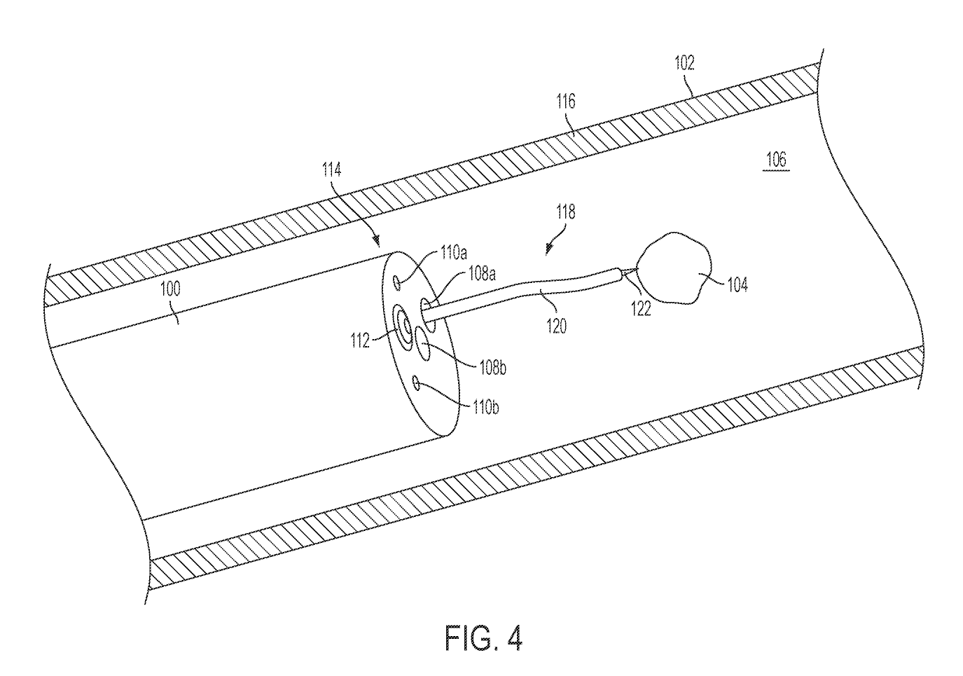

The various embodiments of electrodes described in the present specification, e.g., the electrodes 24a,b may be configured for use with an electrical ablation device 20 comprising an elongated flexible shaft to house the electrodes 24a,b, for example. The electrodes 24a,b are free to extend past a distal end of the electrical ablation device 20. The flexible shaft comprises multiple lumen, channels, or catheters formed therein to slidably receive the electrodes 24a,b. The flexible sheath 26 extends longitudinally from the handle 28 portion to the distal end. The handle 28 portion comprises multiple slide members received in respective slots defining respective walls. The slide members are coupled to the respective electrodes 24a,b. The slide members are movable to advance and retract the electrode 24a,b. The electrodes 24a,b, may be independently movable by way of the respective slide members. The electrodes 24a,b may be deployed independently or simultaneously. The electrical ablation device 20 comprising an elongate flexible shaft to house multiple electrodes and a suitable handle is described with reference to FIGS. 4-10 in commonly owned U.S. patent application Ser. No. 11/897,676 titled “ELECTRICAL ABLATION SURGICAL INSTRUMENTS,” filed Aug. 31, 2007, the entire disclosure of which is incorporated herein by reference in its entirety.

It will be appreciated that the electrical ablation device 20 may be introduced inside a patient endoscopically, transcutaneously, percutaneously, through an open incision, through a trocar, through a natural orifice, or any combination thereof. In one embodiment, the outside diameter of the electrical ablation device 20 may be sized to fit within a channel of the endoscope 12 and in other embodiments the outside diameter of the electrical ablation device 20 may be sized to fit within a hollow outer sleeve, or trocar, for example. The hollow outer sleeve or trocar can be inserted into the upper gastrointestinal tract of a patient and may be sized to also receive a flexible or rigid endoscopic portion of an endoscope (e.g., gastroscope), similar to the endoscope 12 described in FIG. 1.

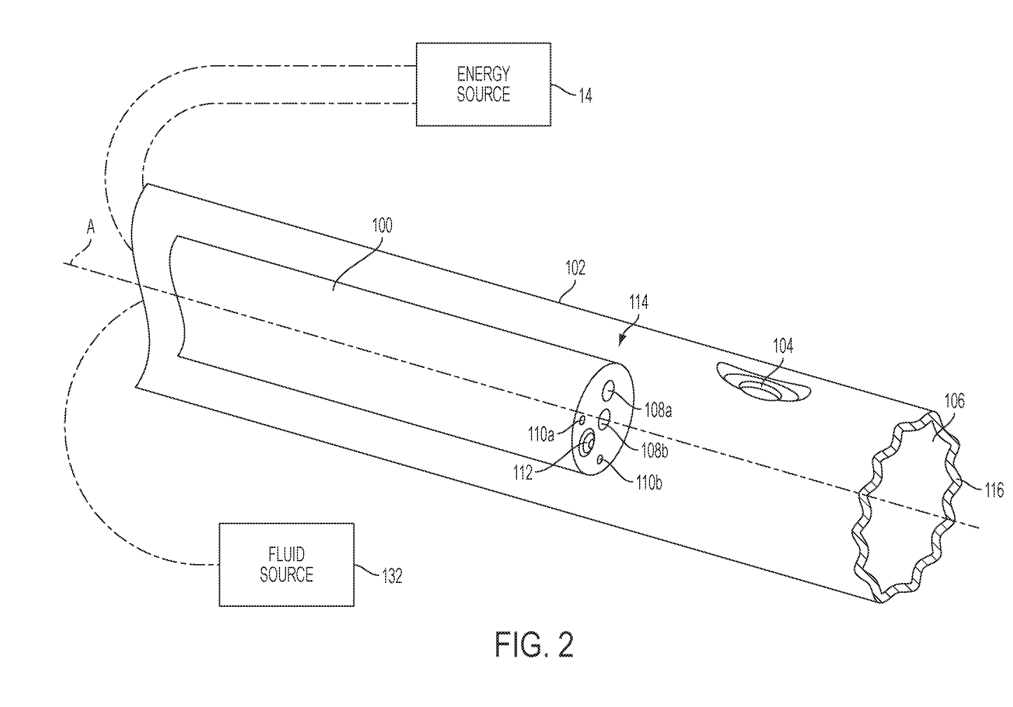

FIGS. 2-9 illustrate one embodiment of a sequence for removing or treating tissue in a tissue treatment region using a pulsed DC electroporation tissue ablation treatment technique. In the embodiment illustrated in FIGS. 2-9, the undesirable tissue is in the form of a sessile polyp 104 growing on the interior portion 106 of the wall 116 of the colon 102. Thus, the tissue treatment region for purposes of this embodiment is the interior portion 106 of the colon 102 surrounding the region proximal to and including the polyp 104. As previously discussed, polyps can be difficult to remove or treat because they are hard to reach. Sessile polyps in particular are difficult to remove or treat due to their low profile and thus are difficult to lasso with a snare in attempt to surgically remove them. Destroying the polyp 104 on the interior portion 106 of the wall 116 of the colon 102 risks rupturing or perforating the colon wall 116, which could be deadly. As previously discussed, DC treatment of tissue (electroporation) is a fast effective way to destroy undesired tissue such as diseased cells, which in this embodiment are cells forming the polyp 104. If a sufficiently high electric field (V/cm) is applied to the polyp 104, cellular structures of the polyp 104 exposed to such electric fields, or higher, will be destroyed while minimizing or eliminating thermal damage to the surrounding healthy tissue of the colon 102, without rupturing or tearing the wall 116 of the colon 102.

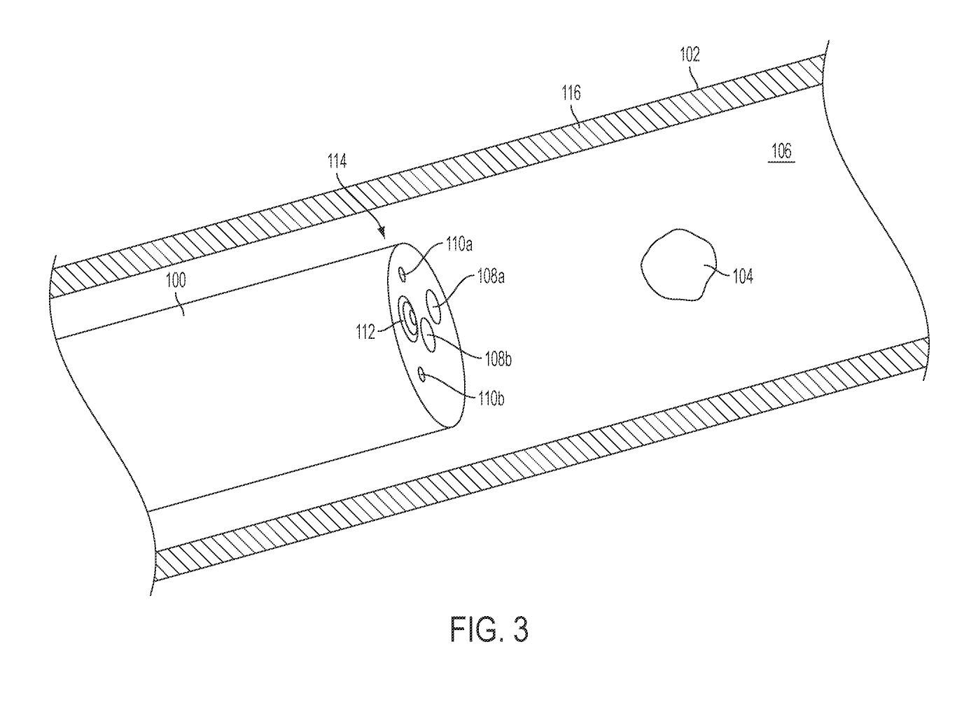

FIG. 2 illustrates an endoscope 100 partially introduced into the colon 102 of a patient for the treatment of a polyp 104 growing on a wall 116 of the colon 102. The colon 102 is illustrated as being transparent for conciseness and clarity. The polyp 104 is growing on an interior portion 106 of the wall 116 of the colon 102. In the illustrated embodiment, the endoscope 100 is a dual channel endoscope. As shown at the distal end 114 of the endoscope 100, the endoscope 100 comprises a first channel 108a and a second channel 108b that extend along a longitudinal axis A of the endoscope 100. It will be appreciated that other endoscopes having a single channel or more than two channels may be employed without limitation. The two channels 108a,b may be instrument or auxiliary channels and are suitable for receiving surgical instruments therethrough. The endoscope 100 also comprises illumination sources 110a,b and an optical viewing port 112 comprising an objective lens. Although the polyp 104 may be representative of any type of undesirable tissue, in this particular example, the polyp 104 is representative of a sessile polyp. FIG. 3 illustrates the wall 116 of the colon 102 in cross-section and the endoscope 100 partially introduced in the colon.

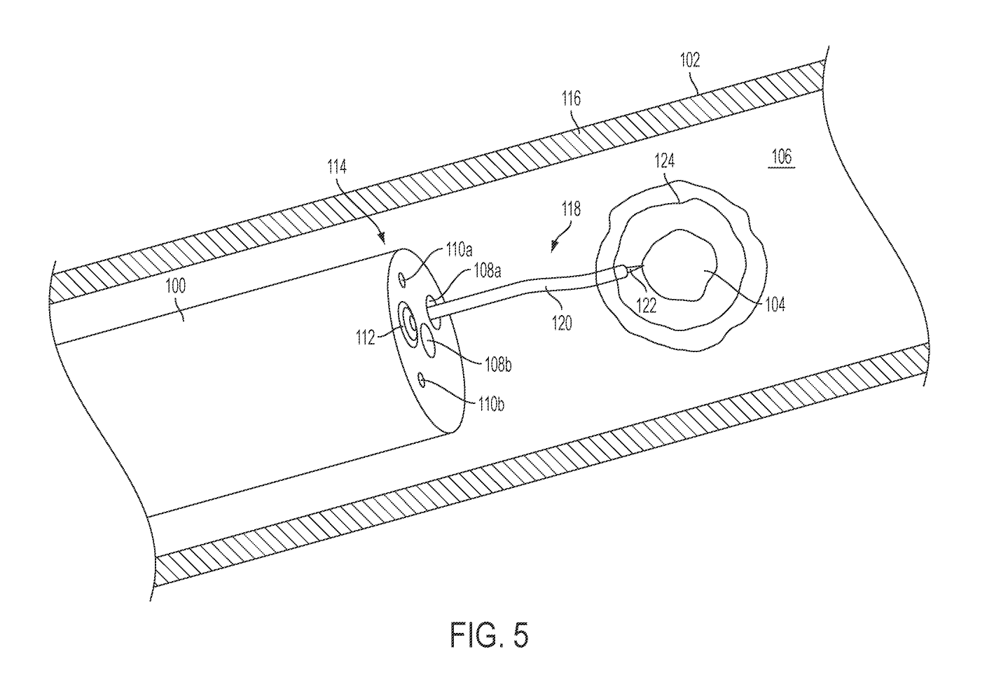

FIG. 4 illustrates one embodiment of an injector catheter electrode 118 introduced into the treatment region proximate to the polyp 104. The injector catheter electrode 118 is advanced through the first channel 108a of the endoscope 100. The distal end of the injector catheter electrode 118 comprises an injection needle 122, which is made from a suitable conductive material such that the injection needle 122 also serves as an electrode. In one embodiment, the injector catheter electrode 118 defines an electrically conductive hollow channel for communicating a fluid from a fluid source 132 (e.g., a supply of air, gas, liquid, saline, and the like) to the treatment site, or in one implementation, for creating a vacuum at the treatment site. In one embodiment, the fluid is electrically conductive, e.g., saline. An electrically insulative sleeve 120 is formed around the injector catheter electrode 118 to prevent electrical short circuits within the channel 108a. The injection syringe is filled with saline or similar fluid for injecting into the polyp 104 via the injector catheter electrode 118 as shown in FIG. 5, which illustrates the injector catheter electrode 118 inserted into the polyp 104 where saline is injected to form a bleb 124 (e.g., a pocket of saline or a blister filled with saline), which lifts or raises the polyp 104 away from the interior portion 106 of the wall 116 of the colon 102. Furthermore, the saline injected into the polyp 104 forming the bleb 124 is electrically conductive and acts as an electrode when energized by the energy source 14.

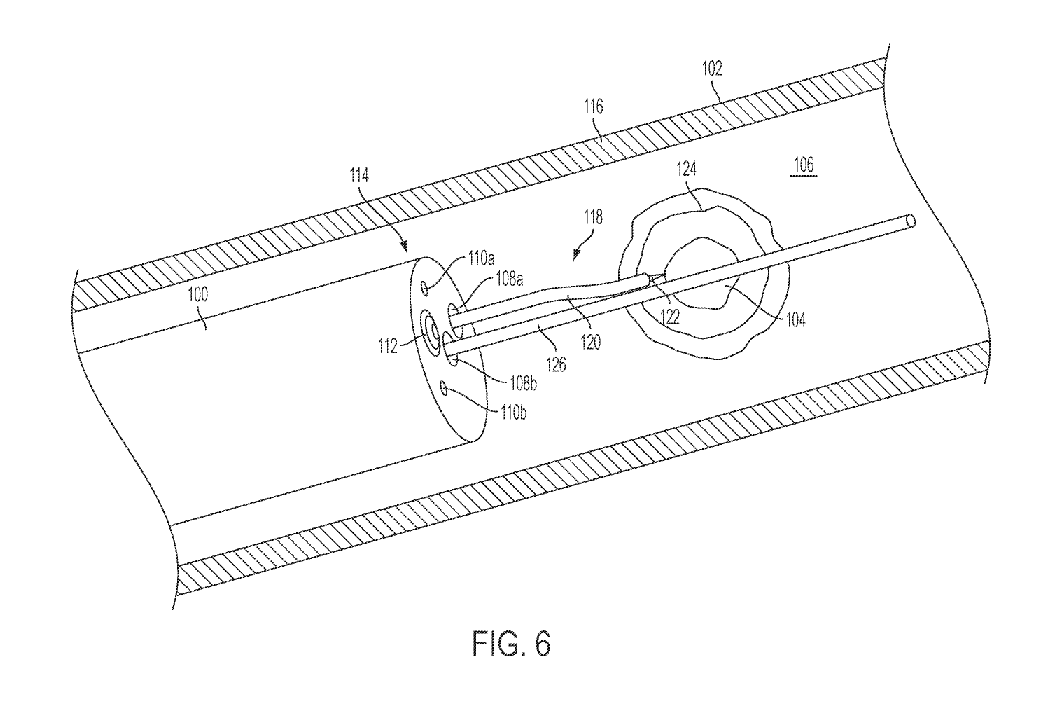

As subsequently discussed with reference to FIGS. 6-8 an electrically conductive balloon electrode 128 is introduced to the tissue treatment region via the second channel 108b and is inflated inside the colon 102, making contact with the polyp 104. Thus, the conductive balloon 128 acts as the other electrode to complete the circuit and electricity can be conducted from the first electrode defined by the injection needle 122 through the bleb 124 and the polyp 104 and through balloon electrode back to the energy source 14. The proximal ends of the injector catheter electrode 118 and the balloon electrode 128 are configured to couple to the fluid source 132, such as an injection syringe, or similar fluid source, and to corresponding electrical conductors 18a,b, handpiece 16, activation switch 62, and energy source 14 (as described herein with reference to FIG. 1).

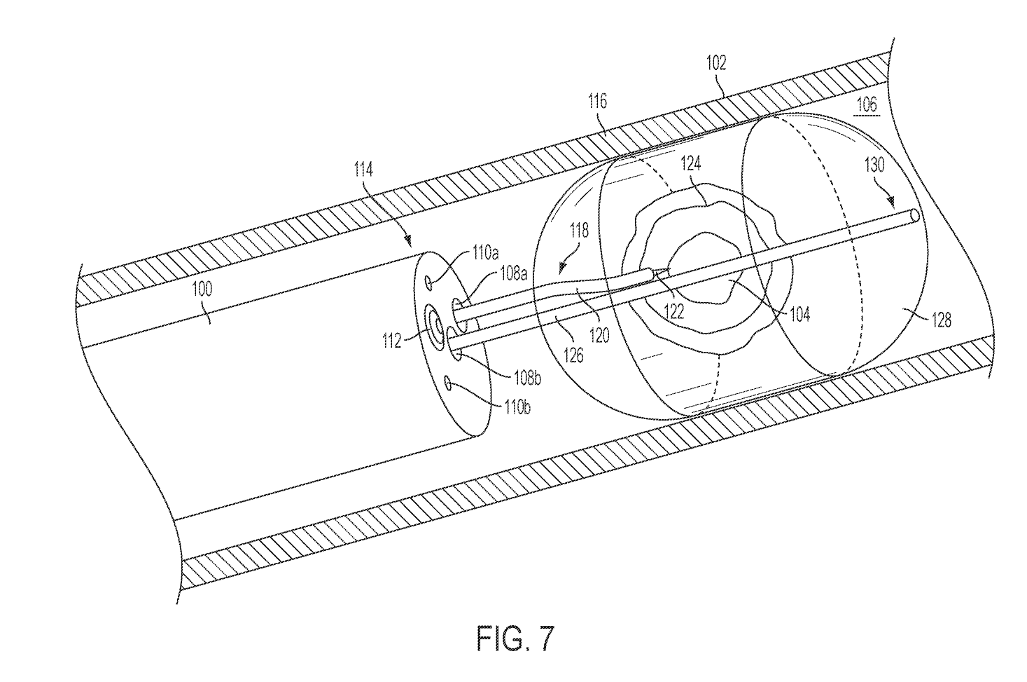

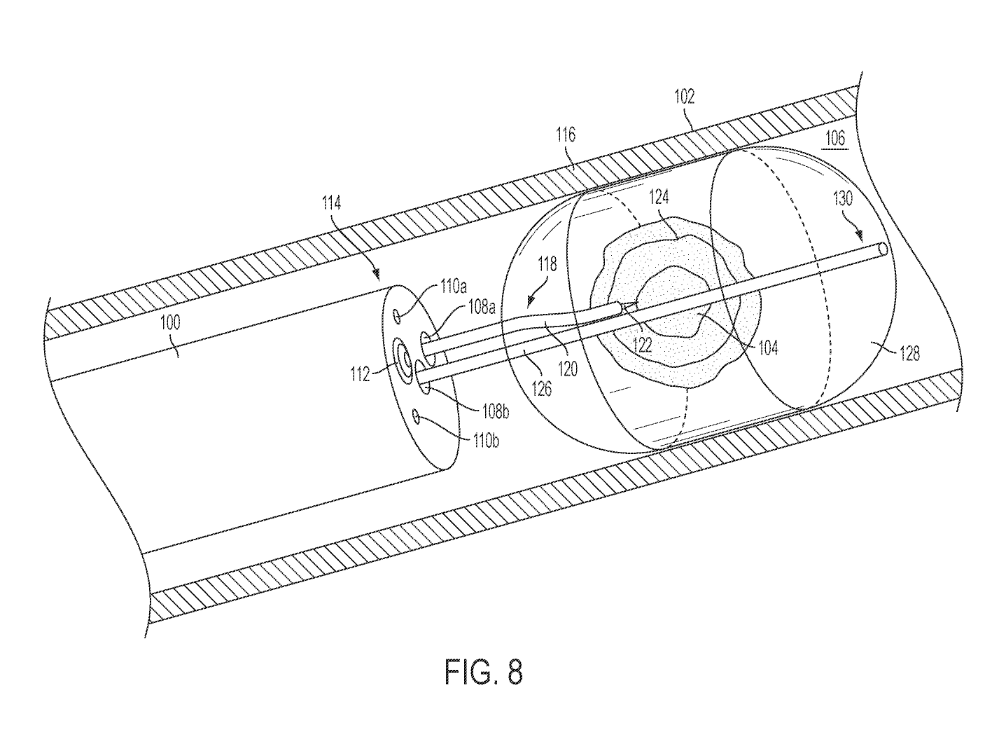

FIG. 6 illustrates one embodiment of a balloon catheter 126 introduced into the treatment region proximate the polyp 104. The balloon catheter 126 is advanced through the second channel 108b of the endoscope 100. The distal end of the balloon catheter 126 is extended past the polyp 104 site. The proximal end of the balloon catheter 126 (not shown) is coupled to the fluid source 132 (e.g., an air and/or liquid supply) used for inflating the balloon 128. Once the distal end 130 of the balloon catheter 126 is extended past the polyp 104 site, a balloon electrode 128 is inflated to substantially fill the inner lumen of the colon 102 and functions as the other electrode. In one embodiment, the inflatable portion of the balloon electrode 128 is formed of a conductive elastomer (e.g., conductive rubber) suitable for coupling to the energy source 14 via an electrically conductive terminal located at the proximal end of the balloon catheter 126 and an electrically conductive wire. Once inflated, the elastomeric properties of the balloon electrode 128 conform to the internal walls 106 of the cavity defined by the colon 102 and contact the polyp 104/bleb 124. As shown in FIG. 7, the illustrated embodiment of the conductive elastomer portion of the balloon electrode 128 acts as the second electrode to conduct electricity from the injection needle 122 electrode through the bleb 124, the polyp 104, the balloon electrode 128 and back to the energy source 14. In one embodiment, the energy source 14 delivers electrical pulses to the bleb 124 and the polyp 104 tissue within the internal 106 walls 126 of the colon 120 cavity to induce irreversible electroporation in the cellular structure of the polyp 104. FIG. 8 shows a change in the bleb 124 and the polyp 104 after the irreversible electroporation treatment is applied.

In one embodiment, the conductive elastomer of the balloon electrode 128 may be fabricated from or may comprise an electrically conductive material suitable for conducting electrical energy from the energy source 14 (as described herein with reference to FIG. 1) to the internal cavity of the colon 102 sufficient to induce irreversible electroporation to the tissue of the polyp 104 and the bleb 124 within the cavity. In one embodiment, the electrically conductive elastomer material of the balloon electrode 128 may comprise silicone, fluorosilicone, EPDM rubber (ethylene propylene diene Monomer (M-class) rubber), a type of synthetic rubber, fluorocarbon-fluorosilicone binder with a filler of pure silver, silver-plated copper, silver-plated aluminum, silver-plated nickel, silver-plated glass, nickel plated graphite, or unplated graphite particles, for example. Conductive elastomers may be formed by infiltrating an elastomeric matrix with electrically conductive filler materials such as silver, gold, copper, nickel, graphite, or aluminum, to produce a hybrid material having the elastic properties of the elastomeric matrix and the electrically conductive properties of the metallic filler materials (some materials may have volume resistivity values as low as 0.004 Ω-cm, for example). The conductive elastomer may be formed as thin sheets, catheters, and balloons suitable for medical applications. In one embodiment, the conductive elastomer balloon electrode 128 may be fabricated from medical grade polyurethane material comprising at least one electrically conductive coating on an outer surface thereof. In another embodiment, the conductive elastomer balloon electrode 128 may be made from an electrically conductive material. In yet another embodiment, the conductive elastomer balloon electrode 128 may be made from an electrically insulative material, such as the medical grade polyurethane, and inflated with a conductive fluid (e.g., saline) to form the electrically conductive portion of the conductive elastomer balloon electrode 128.

In various embodiments the injection needle 122 electrode or the balloon electrode 128 are coupled to opposite polarities of the energy source 14 (as described herein with reference to FIG. 1). Thus, if the injection needle 122 electrode is coupled to the anode (+) of the energy source 14, the balloon electrode 128 is coupled to the cathode (−) of the energy source 14, and vice-versa. It will be appreciated that, in one embodiment, the polarity of the injection needle 122 and the balloon electrode 128s may be reversed by reversing the output polarity of the energy source 14. In one embodiment, the injection needle 122 electrode is coupled to the anode (+) of the energy source 14 and the balloon electrode 128 is coupled to the cathode (−) of the energy source 14. In this configuration, the polyp 104/bleb 124 is now electrical load of the energy source 14. An electric field anywhere in the range of about 80,000 V/m to about 5,000,000 V/m is sufficient to destroy tissue located between the injection needle 122 and the balloon electrode 128s in about one second. Accordingly, the application of an electric field in this range will destroy the polyp 104 with minimal or no damage to the healthy tissue around the polyp 104 and without rupturing the wall 116 of the colon 102. The applied electric field is discussed in more detail in FIG. 9.

In one embodiment, treatment of the polyp 104 may be effected by applying a sequence of electrical pulses to the injection needle 122 electrode and applying a ground potential to the balloon electrode 128. It will be appreciated that, in one embodiment, the polarity may be reversed such that the sequence of electrical pulses is applied to the balloon electrode 128 and the ground potential is applied to the injection needle 122 electrode. It can be further appreciated that, in one embodiment, the sequence of electrical pulses can be applied differentially between (1) the injection needle 122 electrode and (2) the balloon electrode 128. In one embodiment, the sequence of pulses have amplitudes in the range of about ±100 to about ±10,000VDC, pulse lengths (e.g., pulse width, pulse duration) in the range of about 1 μs to about 100 ms, and frequencies in the range of about 1 Hz to about 10,000 Hz.

FIG. 9 is a graphical representation of electric field strength in volts per meter (V/m) developed across the polyp 104 when the injection needle 122 electrode is energized by the energy source 14 (as described herein with reference to FIG. 1) and the balloon 128 (FIGS. 6-8) electrode acts as a return. As illustrated in FIG. 9, the polyp 104 and the colon wall 116 are shown in cross-section. The polyp 104 is raised from the colon wall 116 by the saline injected by the injection needle 122 and is treated with DC pulses. The horizontal and the vertical axes represent distance in meters (m) with the center defined at (0,0) where the tip of the injection needle 122 is inserted into the polyp 104. FIG. 9 also illustrates a graph of electric field strength developed when the injection needle 122 electrode, inserted into the polyp 104/bleb 124, and the balloon electrode 128, inflated proximate the polyp 104/bleb 124, are energized by the energy source 14. A vertical scale 130 shown to the right of the graph represents the electric field strength in a range from a minimum of about 80,000V/m (bottom) to a maximum of about 5,000,000V/m (top). Irreversible electroporation energy in this range of electric field strength (e.g., about 80,000V/m to about 5,000,000V/m) are suitable for efficient and effective treatment of medical conditions that require the ablation of undesirable tissue from a localized region (i.e., in the case of the treatment of sessile polyps 104). It will be appreciated that other electric field strength may be developed to render effective irreversible electroporation ablation therapy. Accordingly, the embodiments described herein should not be limited in this context.

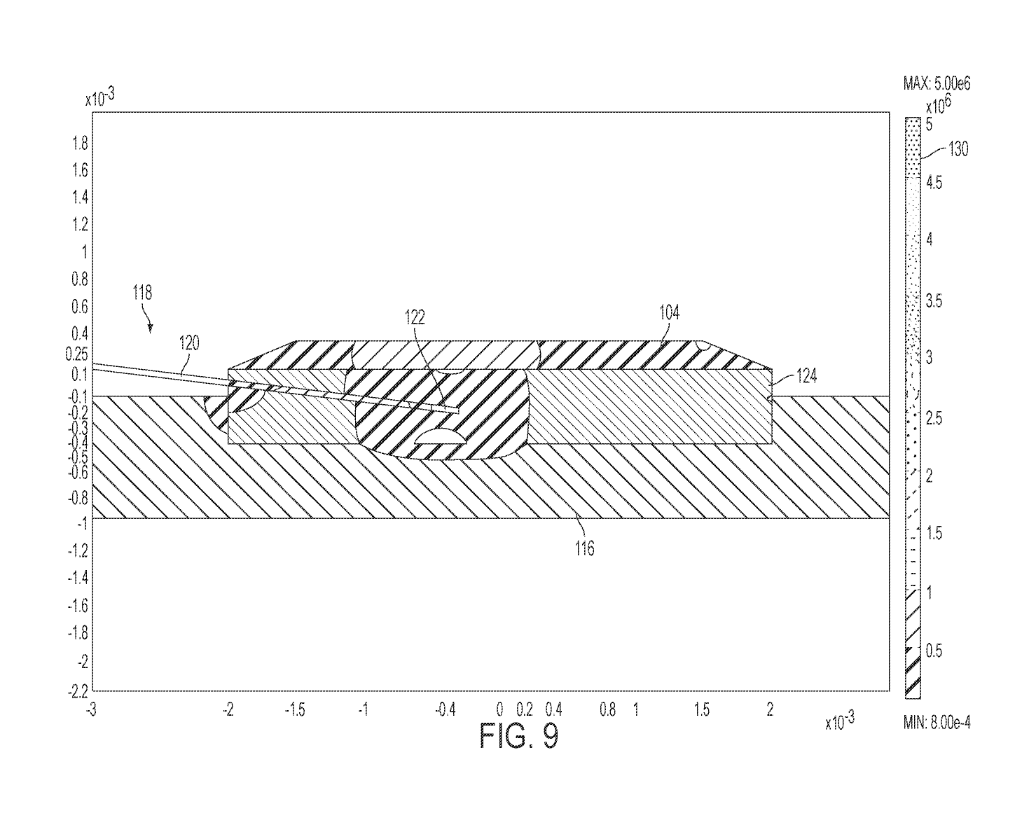

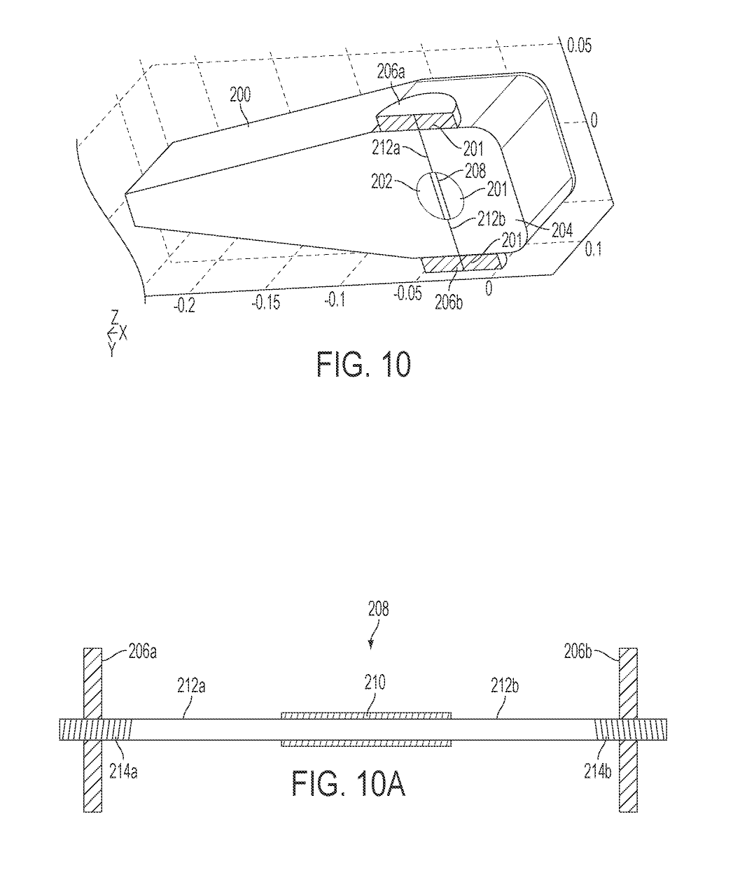

FIGS. 10-12 illustrate one embodiment of an ablation device for treating tumors embedded in a larger mass of tissue. In one embodiment, the ablation device is adapted and configured for the treatment of liver tumors. In one particular implementation, the ablation technique is employed in the treatment of liver tumors that are in the range of 3-5 cm in diameter and that are embedded deep in the liver tissue. FIG. 10 is a cross-sectional sectional view of a liver 200 showing a tumor 202 embedded in a single lobe 204 of the liver 200 and one embodiment of an ablation device 201 piercing through the tumor 202 and clamping the single lobe 204. The ablation device 201 comprises first and second electrodes 206a and 206b that are configured for placement on the outer surface of the liver 200 and are coupled to the energy source 14 (as described herein with reference to FIG. 1). In one embodiment, the first and second electrodes 206a,b have a plate-like shape, e.g., substantially thin and flat where the surface area is grater than the thickness to allow the plates to apply a compression force on the single lobe 204 of the liver 200. The first and second electrodes 206a,b may be any suitable shape including, for example, polygonal, circular, triangular square, rectangular. The first and second electrodes 206a,b have a threaded opening to receive threaded ends of a third electrode 208. The third electrode 208 penetrates the liver 200 and the tumor 202 and is also coupled to the energy source 14. The third electrode 208 comprises an electrically conductive portion 210 and non-conductive portions 212a, 212b. In the embodiment illustrated in FIG. 10, the electrically conductive portion 210 of the third electrode 208 is shown inserted through the tumor 202. In one embodiment, the first 206a, second 206b, and third electrodes 208 are coupled to corresponding electrical conductors 18a,b, handpiece 16, activation switch 62, and energy source 14 (as described herein with reference to FIG. 1).

FIG. 10A illustrates one embodiment of the ablation device 201 shown in FIG. 10. In one embodiment, the third electrode 208 has first and second threaded ends configured to threadably engage the first and second threaded openings formed in the respective first and second electrodes 206a,b. The third electrode 208 comprises a conductive portion between the first and second threaded ends and electrically insulative portions between the conductive portion and the first and second threaded ends, wherein the conductive portion is electrically isolated from the first and second electrodes. In the embodiment shown in FIG. 10A, the non-conductive portions 212a,b of the third electrode 208 may be constructed of an electrically insulative material such as ceramic and a center conductive portion 210 may be constructed of an electrically conductive material such as medical grade stainless steel, copper, gold, aluminum, nickel, brass. In one embodiment, the center conductive portion 210 of the third electrode 208 may be coated onto the outer surface of a non-conductive portion forming a conductive layer over the non-conductive body of the third electrode 208. Any suitable method of applying the conductive layer coating may be employed including, for example, any suitable application technique that promotes good adhesion of the conductive material to the non-conductive base material of the body of the third electrode 208. The conductive material may be applied to the non-conductive base material of the body of the third electrode 208 using suitable material application techniques, such as, for example, coating, dipping, printing, spraying, brushing, drying, melting, laser curing, anodizing, electroplating, electroless chemical deposition, sintering, fused curing, physical vapor deposition (PVC), chemical vapor deposition (CVD), thermal spray, thick film high velocity oxygen fuel (HVOF) plasma, and any other suitable material application techniques. In other embodiments, one non-conductive portion 212a may be attached to one end of the conductive portion 210 and another non-conductive portion 212b may be attached to another end of the conductive portion 210. The non-conductive portions 212a,b may be attached to the conductive portion 210 using any suitable methods for joining the three components such as, for example, bolting, screwing, welding, crimping, gluing, bonding, brazing, soldering, press fitting, riveting, heat shrinking, heat welding, ultrasonic welding, or any other suitable method. The overall length of the conductive portion 210 of the electrode 208 is selected to fit the size of the tumor 202.

With reference to both FIGS. 10 and 10A, in one embodiment the non-conductive portions 212a,b on either end of the conductive portion 210 may be threaded so as to threadably engage first and second threaded openings 214a, 214b formed in the first and second electrodes 206a,b, respectively. In use, once each of the electrodes 206a,b is placed on either side of the liver 200 above and below the tumor 202, the third electrode 208 is advanced through the first opening 214a formed in the first electrode 206a, through the tumor 202, and then through the second opening 214b formed in the second electrode 206b. In the embodiment comprising the thread mechanism, the third electrode 208 may be used to compress the liver 200 by rotating the third electrode 208 and threadably engaging the outer first and second electrodes 206a,b to cause the first and second electrodes 206a,b to advance toward each other.

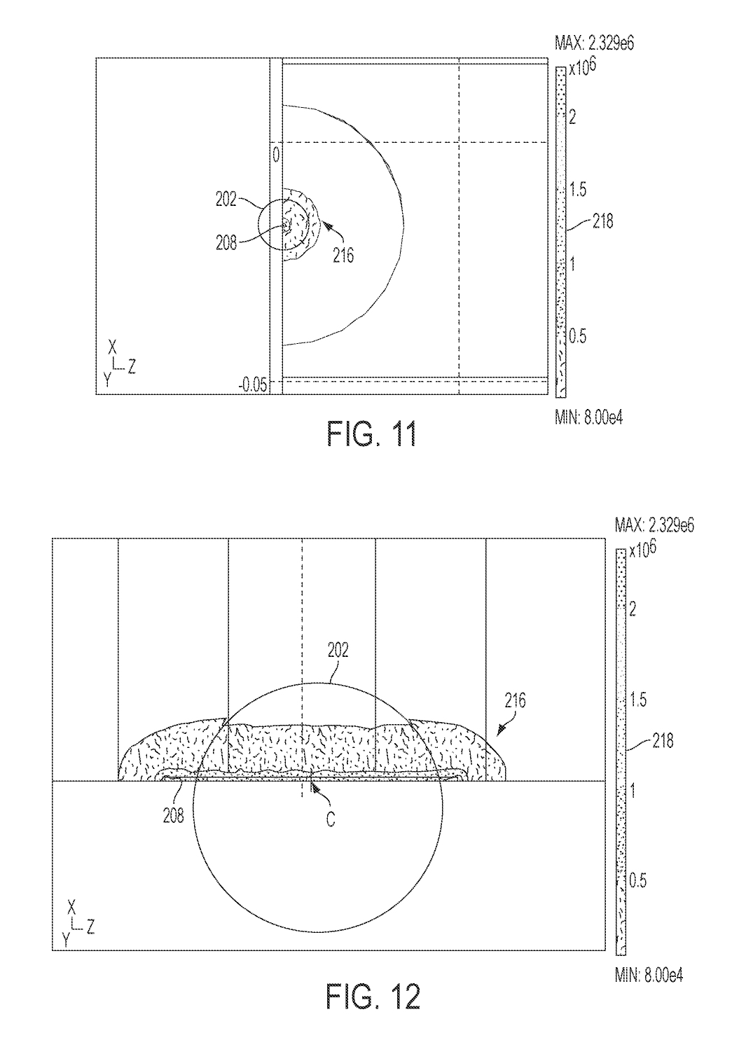

Electrically conductive wires are connected to each of the first, second, and third electrodes 206a, 206b, and 208, respectively, using any suitable method. The electrically conductive wires are connected to the energy source 14 (as described herein with reference to FIG. 1) in any suitable configuration. In one embodiment, the first and second electrodes 206a,b are connected to the ground terminal (or negative potential terminal) of the energy source and the third electrode 208 is connected to the positive potential terminal of the energy source 14. When the third electrode 208 is energized with a sequence of electrical pulses, an electric field is created around the tumor 202 as discussed in more particularity herein with reference to FIGS. 11 and 12. In one embodiment, treatment of the tumor 202 may be effected by applying a sequence of electrical pulses to the third electrode 208 and applying a ground potential to the first and second electrodes 206a,b. It will be appreciated that, in one embodiment, the polarity may reversed such that the sequence of electrical pulses are applied to the first and second electrodes 206a,b and the ground potential is applied to the third electrode 208. It can be further appreciated that, in one embodiment, the sequence of electrical pulses can be applied differentially between (1) the first and second electrodes 206a,b and (2) the third electrode 208. In one embodiment, the sequence of electrical pulses have amplitudes in the range of about ±100 to about ±10,000VDC, pulse lengths in the range of about 1 μs to about 100 ms, and frequencies in the range of about 1 Hz to about 10,000 Hz. To enhance the effectiveness of the ablation treatment, the lobe liver 204 of the liver 200 may be compressed by rotating the third electrode 208 and threadably advancing the outer first and second electrodes 206a,b toward each other and then reapplying the sequence of electrical pulses. This process can be repeated until the tumor is treated.

FIGS. 11 and 12 are graphical representations of the electric field applied to the treatment region showing necrotic region formed in the ablation zone 216 around the tumor 202 when the third electrode 208 is energized with a pulsed positive potential and the first and second electrodes 206a,b are connected to ground potential. FIG. 11 illustrates an end view of the ablation zone 216 and FIG. 12 illustrates a side view of the ablation zone 216. The horizontal and the vertical axes represent distance in meters (m) with the center defined at (0,0) where the center C of the third electrode 208 is located. FIGS. 11 and 12 graphically illustrate the electric field strength developed when the third electrode 208 inserted into the tumor 202 is energized by the energy source 14 relative to the first and second electrodes 206a,b located outside the liver 200. A vertical scale 218 shown to the right of the graph represents the electric field strength in a range from a minimum of about 80,000V/m (bottom) to a maximum of about 2,329,000V/m (top). Irreversible electroporation energy in this range of electric field strength (e.g., about 80,000V/m to about 2,329,000V/m) is suitable for efficient and effective treatment of medical conditions that require the ablation of undesirable tissue from a localized region (i.e., in the case of the treatment of liver tumors 202). It will be appreciated that electric fields of varying strength may be developed to render effective irreversible electroporation ablation therapy. Accordingly, the embodiments described herein should not be limited in this context.

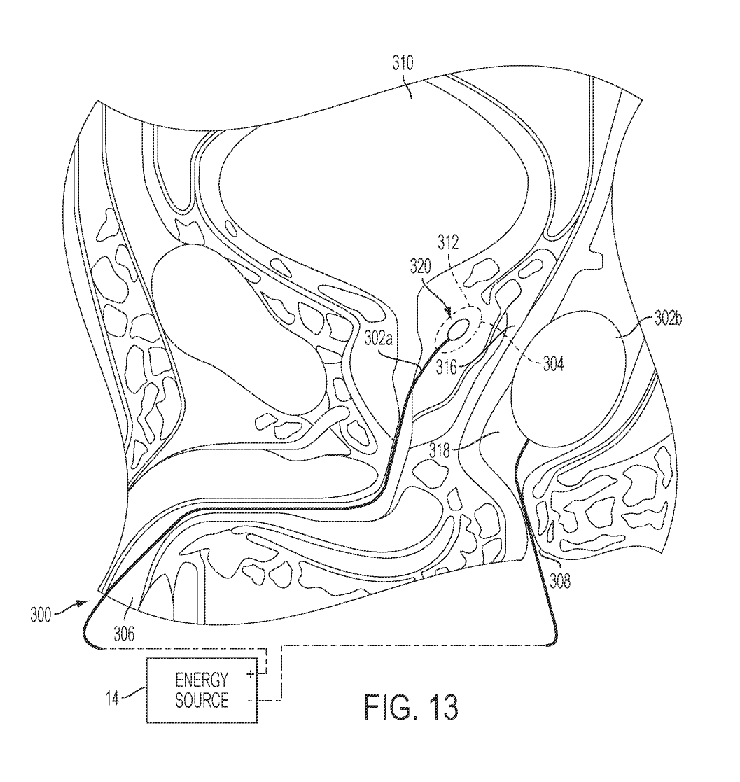

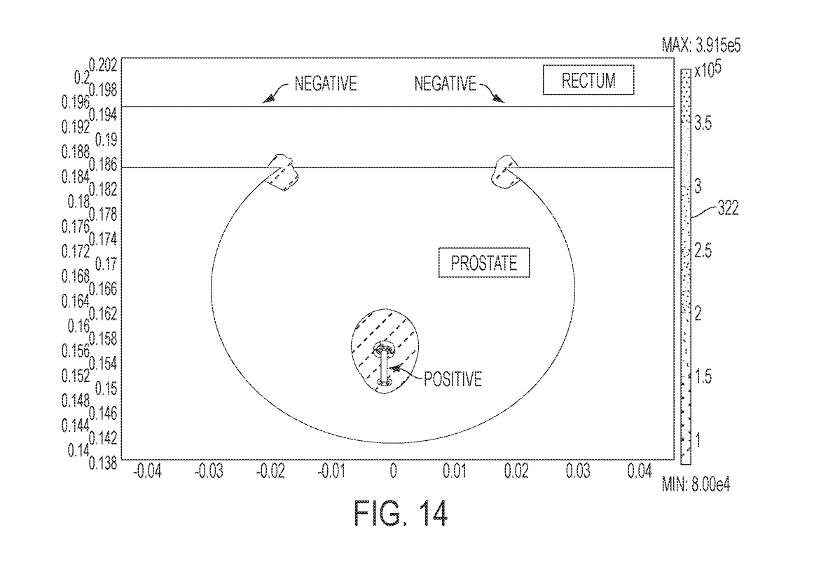

FIGS. 13 and 14 illustrate implementations of thermal ablation techniques for ablating hyperplastic cells in the prostrate gland to reduce the size of the prostrate. FIG. 13 is a cross-sectional view of the male pelvis and one embodiment of an electrical ablation system 300 for ablation treatment of the prostate by applying high voltage DC pulses to the treatment region. The electrical ablation system 300 in accordance with the disclosed embodiments provides improved electrical ablation of hyperplastic cells in the prostrate gland to reduce the size of the prostrate using DC pulses supplied by an energy source. One or more than one electrode is positioned directly into the prostratic lobe and one or more than one electrode is positioned outside the prostratic lobe. When the electrodes are energizes with a pulsed electric potential, the hyperplastic cells are ablated to reduce the size of the prostrate.

With reference to FIG. 13, the electrical ablation system 300 comprises an electrical ablation device 302 comprising at least two electrodes 302a,b coupled to the energy source 14 (as described herein with reference to FIG. 1). The electrical ablation system 300 may be adapted for use in conjunction with the electrical ablation system 10 described in FIG. 1. The electrodes 302a,b are configured to be positioned within internal body lumens or cavities and, in one embodiment, may be configured for use in conjunction with the flexible endoscope 12 also described in FIG. 1. The electrodes 302a,b are configured to couple to corresponding electrical conductors 18a,b, handpiece 16, activation switch 62, and energy source 14 (as described herein with reference to FIG. 1). In one embodiment, the first electrode 302a is a catheter electrode comprising a wire or flexible conductive tube that may be introduced into the urethra 306 and advanced into the prostate 304 by puncturing through the urethra 306. The electrode 302a is finally positioned in the prostate 304 treatment zone 320 proximal to the bladder 310. The first electrode 302a may be located directly into the prostate 304 using well known fluoroscopy, ultrasonic guidance, or a cystoscope, for example. The second electrode 302b in the form of a conductive balloon electrode may be introduced into the anus 308 and advanced to a location proximate to but outside and directly behind the prostate 304 in the rectum 318. The balloon electrode 302b may be introduced using an ultrasound probe and then inflated with saline once inside the rectum 318. The first electrode 302a has a much smaller surface area relative to the trans-anally placed second balloon electrode 302b. The conductive balloon electrode 302b may be similar to in operation and construction to the balloon electrode 128 described herein with reference to FIGS. 7 and 8. The first electrode 302a may be connected to the positive (+) terminal of the energy source 14 and the second electrode 302b may be connected to the negative (−) terminal of the energy source 14. In one embodiment, the energy source 14 may be configured as a high-voltage DC electric pulse generator. The activation switch 62 portion of the handpiece 16, as shown in FIG. 1, can be used to energize the electrical ablation system 300 to ablate the hyperplastic cells in the prostrate 304 by DC pulses supplied by the energy source 14 and delivered through the electrodes 302a,b as described in FIG. 14 below.

Treatment of the prostate may be effected by applying a sequence of electrical pulses to the catheter electrode 302a and applying a ground potential to the balloon electrode 302b. It will be appreciated that, in one embodiment, the polarity may be reversed such that the sequence of electrical pulses is applied to the balloon electrode 302b and the ground potential is applied to the catheter electrode 302a. It can be further appreciated that, in one embodiment, the sequence of electrical pulses can be applied differentially between (1) the catheter electrode 302a and (2) the balloon electrode 302b. In one embodiment, the sequence of pulses have amplitudes in the range of about ±100 to about ±10,000VDC, pulse lengths (e.g., pulse width, pulse duration) in the range of about 1 μs to about 100 ms, and frequencies in the range of about 1 Hz to about 10,000 Hz.

FIG. 14 is finite element model of the electric field created in the prostrate 304 when the electrodes 302a,b are energized. With the electrodes 302a,b positioned as described in FIG. 13, when the DC pulses are applied to the positive electrode 302a located directly in the prostrate 304a necrotic zone 312 is created around the positive electrode 302a. Although a small amount of necrosis 314 also occurs at the prostratic/rectal interface 316, it will likely not be a clinically significant amount. The applied pulses are in the order of about 3 kVDC with a pulse duration of about 50 μs. Generally, multiple pulses will be applied. As shown in FIG. 14, the horizontal and the vertical axes represent distance in meters (m) with the center defined at by the location of the first electrode 302a located directly in the prostrate 304. FIG. 14 also illustrates a graph of electric field strength developed when the first electrode 302a, which is located directly in the prostrate, is energized with a positive potential relative to the second electrode 302b, which is located in the rectum 318. A vertical scale 322 shown to the right of the graph represents the electric field strength in a range from a minimum of about 80,000V/m (bottom) to a maximum of about 391,500V/m (top). Irreversible electroporation energy in this range of electric field strength (e.g., about 80,000V/m to about 391,500V/m) is suitable for efficient and effective treatment of medical conditions that require the ablation of undesirable tissue from a localized region (i.e., in the case of the treatment of the hyperplastic tissue in the prostrate 304). It will be appreciated that other electric field strength may be developed to render effective irreversible electroporation ablation therapy. Accordingly, the embodiments described herein should not be limited in this context.

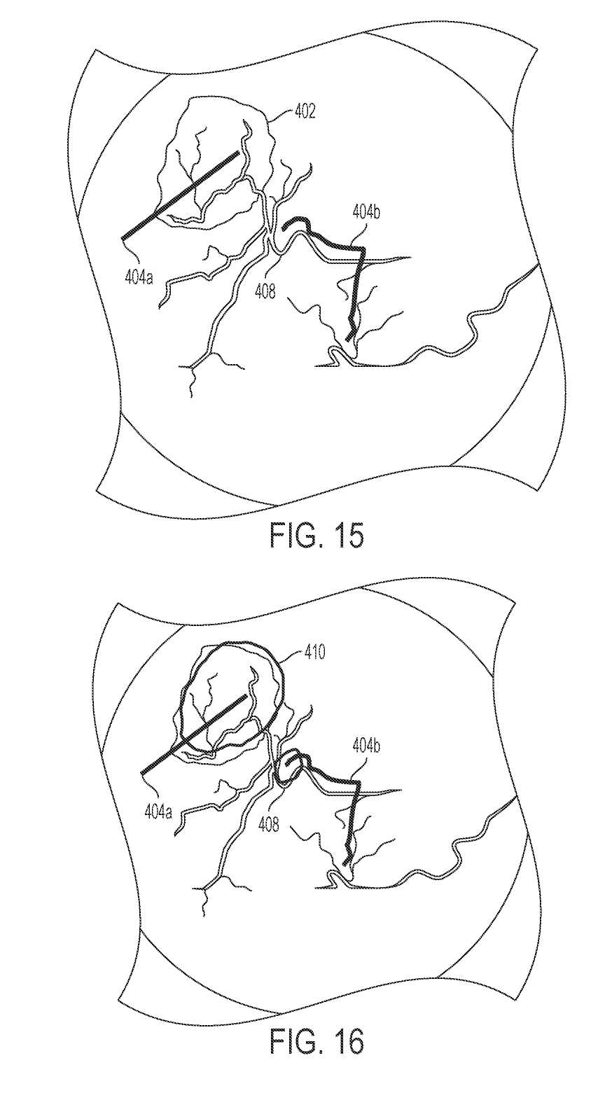

FIGS. 15 and 16 illustrate a hepatic tumor before and after treatment by the application of high voltage DC pulses with an ablation system. The tumor 402 is representative of hepatocellular cancer (HCC) and colorectal liver metastases (CRLM), two of the most common hepatic malignancies. Several embodiments of electrical ablation techniques are described herein as alternatives to surgical resection for treating these malignant hepatic tumors 402. When surgical resection is not possible (e.g., severely damaged liver from cirrhosis) the ablation technique disclosed herein may be offered to the patient as an alternative along with systemic chemotherapy.

The various embodiments of the ablation techniques to treat hepatic malignant tumors 402 include a combination of high voltage DC pulses with catheters in the hepatic artery to deliver necrosis agents, conductive fluids, and simple electrodes 404a,b inserted into the tumor and the arterial supply. The electrodes 404a,b are coupled to corresponding electrical conductors 18a,b, handpiece 16, activation switch 62, and energy source 14 (as described herein with reference to FIG. 1) to complete the electrical circuit.

FIG. 15 is a radiological image illustrating a first electrode 404a placed in the tumor 402, which is fed by an arterial blood supply, and a separate second electrode 404b placed intravenously through the hepatic artery 408. In one embodiment, multi-electrodes are used in the application of high voltage DC pulses. The second electrode 404b is advanced as far as possible to be near the arterial branches supplying the tumor 402. The electric field created between the two electrodes 404a,b when they are energized by the energy source 14 will cause irreversible damage to the cells of the tumor 402. To achieve such electric field strength, the energy source 14 has to generate a high voltage of about 3 kV.

FIG. 16 is a radiological image illustrating the ablation zone 410 that is outlined by obliterated capillaries 412 from the high voltage DC pulse treatment. The advantage of this approach over placing two electrodes directly in the tumor 402 is to directly treat the cells on the periphery of the tumor 402. In one embodiment, treatment of the hepatic tumor 402 may be effected by applying a sequence of electrical pulses to the first electrode 404a and applying a ground potential to the second electrode 404b. It will be appreciated that, in one embodiment, the polarity may be reversed such that the sequence of electrical pulses is applied to the second electrode 404b and the ground potential is applied to the first electrode 404a. It can be further appreciated that, in one embodiment, the sequence of electrical pulses can be applied differentially between (1) the first electrode 404a and (2) the second electrode 404b. In one embodiment, the sequence of pulses have amplitudes in the range of about ±100 to about ±10,000VDC, pulse lengths (e.g., pulse width, pulse duration) in the range of about 1 μs to about 100 ms, and frequencies in the range of about 1 Hz to about 10,000 Hz.

In another embodiment, a conductive fluid, such as saline, may be injected into the arterial system proximate the second electrode 404b to act as the return electrode and to increase the conductivity of the path to the tumor 402 and therefore increase the electric field near the tumor 402. This kills the blood supply to the tumor 402 prior to killing the tumor 402.

In another embodiment, the tumor 402 may be treated using a combined application of high voltage DC pulses directly to the liver tumor 402 and adjuvant chemoembolization. Percutaneous Ethanol Injection (PEI) and polyvinyl alcohol (PVA) beads are therapeutic procedures that involve administering chemical agents through the hepatic arterial supply to reduce the blood supply to the tumor by embolizing the arterial supply. Since the majority of blood supply to the healthy hepatocytes comes from the portal vein, this method does not compromise the liver. The combination of ablation and chemo-embolization could be described as working from the inside out (ablation) and outside in (chemo-embolization). Thus, in one embodiment, this treatment process includes the combination of the application of high voltage DC pulses to the tumor 402 and chemo-embolization. The application of high voltage DC pulses (electroporation) causes cell necrosis around the electrodes 404a,b without causing irreversible damage to the surrounding structures (e.g., larger blood vessels). The embolizing agent is then able to penetrate the tumor 402 to cause necrosis from the outside in. Hepatic arterial injection (HAI) of chemo drugs also provides an effective way to reduce tumor progression or eliminate tumors.

Accordingly, in yet another embodiment, high voltage DC pulses can be applied to the tumor 402 in combination with systemic electro-chemotherapy such as HAI. As previously discussed, electroporation (the application of high voltage DC pulses to tissue) is a method traditionally used to increase the permeability of the cell wall to molecules. When the DC pulse is applied, the molecules will travel through the pores in the cell wall and remain in the cell after the application of pulses is terminated. The cell may then become necrotic due to the toxicity of the injected molecule (e.g., cystplatin) or an immunological response that causes a systemic failure of the tumor cells. When the DC pulse voltage is increased, irreversible damage of the cell occurs. This is the mechanism of necrosis previously described. Beyond the threshold of irreversible damage, the cells will not be irreversibly damaged but will presumably be made more permeable. When a bolus of chemotherapy drugs is applied either systemically or directly through the hepatic artery, the cells will be more permeable to these molecules. This combination of irreversible damage from the pulses and electro-chemo damage from the drugs will increase the size of the necrotic zone. The increase in size will be in the shape of the arterial supply of the tumor, therefore causing more efficient necrosis.

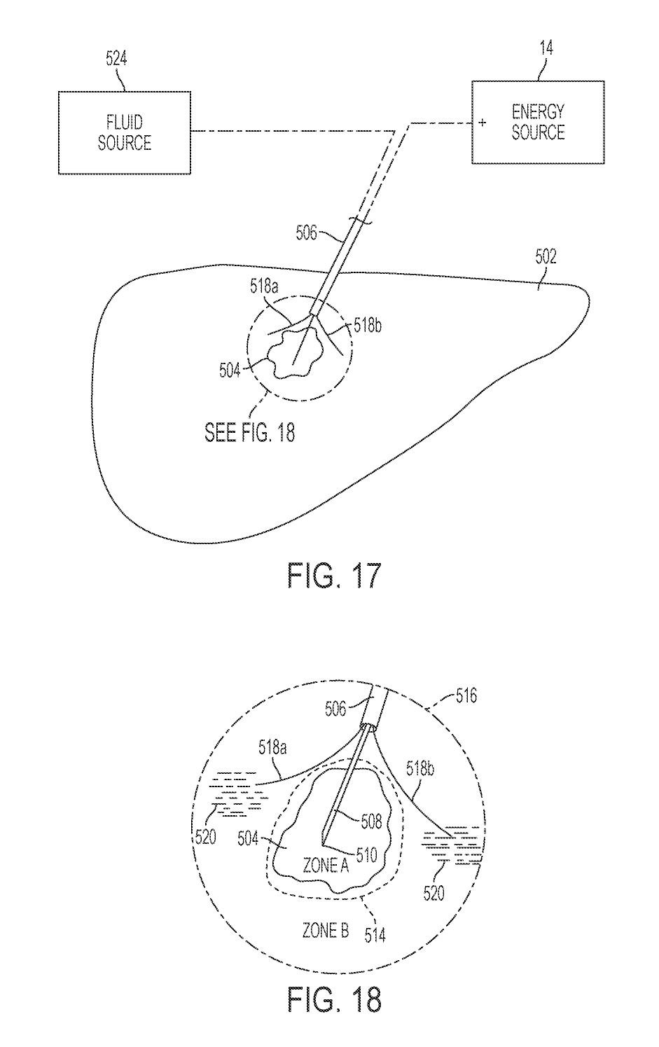

FIGS. 17, 18, 19, 20A-E, and 21 illustrate additional embodiments of devices and methods for debulking tumors and causing a specific systemic response by employing electroporation techniques. As previously discussed, electroporation is a method by which aqueous pathways are formed in the cell plasma membrane by increasing the plasma membrane voltage. Electro-chemotherapy has been used for many years to inject material into the cell cytoplasm (through the pores created by the increased voltage) to cause the destruction of cancer cells. Experimental studies have shown that cellular uptake of DNA led to transfection in vivo and subsequent induction of immune responses. An increase in the plasma membrane voltage can lead to cell necrosis. One embodiment of a method and device for performing the above tasks simultaneously will now be described in connection with FIGS. 17-20.

FIG. 17 shows a liver 502, a tumor 504, and one embodiment of a probe 506 placed into the tumor 504. FIG. 18 is a detailed view of the probe 506 and the tumor 504. When a single electrode 508 is placed in the tumor 504 and another electrode is place elsewhere on the body, the electric field strength will decrease as a function of the distance between the electrodes. FIG. 18 is a graphical representation of an electric field strength E (V/cm) diagram as a function of distance from the center 510 of the central electrode 508. Electric field strength E (V/cm) is shown along the vertical axis and distance d (cm) from the center of the electrode is shown on the horizontal axis. The electric field strength Eₘ (V/cm) is strongest at the location coinciding with the center 510 of the central electrode 508 and decays exponentially 512 as the distance d increases away from the center 510.

In one embodiment, treatment of the tumor 504 may be effected by applying a sequence of electrical pulses to the first electrode 508 and applying a ground potential to a second electrode (not shown). It will be appreciated that, in one embodiment, the polarity may be reversed such that the sequence of electrical pulses is applied to the second electrode and the ground potential is applied to the first electrode 508. It can be further appreciated that, in one embodiment, the sequence of electrical pulses can be applied differentially between (1) the first electrode 508 and (2) the second electrode. In one embodiment, the sequence of pulses have amplitudes in the range of about ±100 to about ±10,000VDC, pulse lengths (e.g., pulse width, pulse duration) in the range of about 1 μs to about 100 ms, and frequencies in the range of about 1 Hz to about 10,000 Hz.

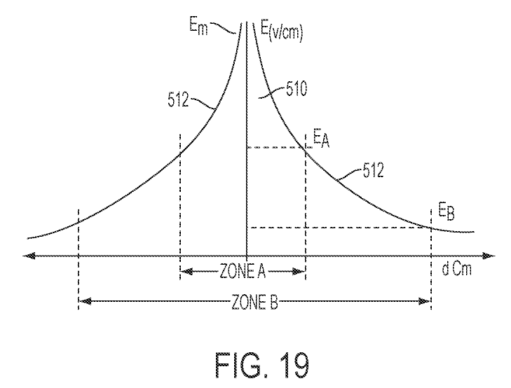

FIG. 19 is a graphical representation of electric field strength in two discernible zones of electric field strength created at distances proximate to the center of an energized electrode 506. With reference to FIGS. 18 and 19, Zone A has been outlined by perimeter 514 envelopes the tumor 504 and has an electric field strength of E_{A} (V/cm). Zone A is outlined as the volume where the electric field strength is above the necrotic threshold. It has been reported that the electric field strength needed to cause cell necrosis is about 700 V/cm. Therefore, the electric field strength within Zone A E_{A} should be about 700 V/cm. Zone B is outlined by perimeter 516 and is located further away from the center 510 of the central electrode 508. Zone B has been outlined as the zone where the field strength will not cause necrosis but will cause poration of the plasma membranes.

With reference to FIGS. 17 and 18, in one embodiment, the probe 506 comprises one or more than one injection needle 518 which are used to inject fluids into the tumor 504. In one embodiment, the injection needle 518 is used to inject a DNA plasmid 520 before the pulses are applied from the energy source 14 (as described herein with reference to FIG. 1). The combination of electrical pulses and DNA plasmid 520 will cause necrosis of the central tumor 504 (debulk) and cause a specific immunological response that destroys the circulation of tumor cells throughout the body. Interleukins would be injected to stimulate a T cell response, which would act directly on cancer cells as well as induce a specific response. This method could be applied to many cancers (e.g., breast) and different combinations of electrodes could be used.

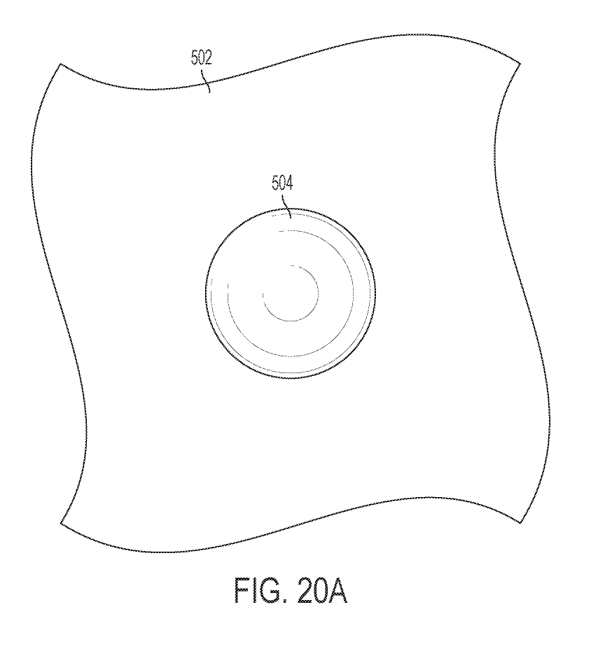

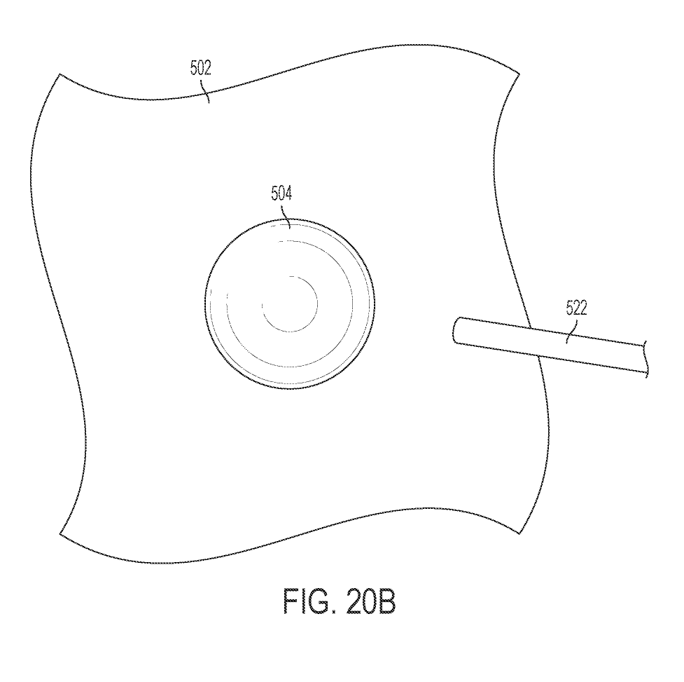

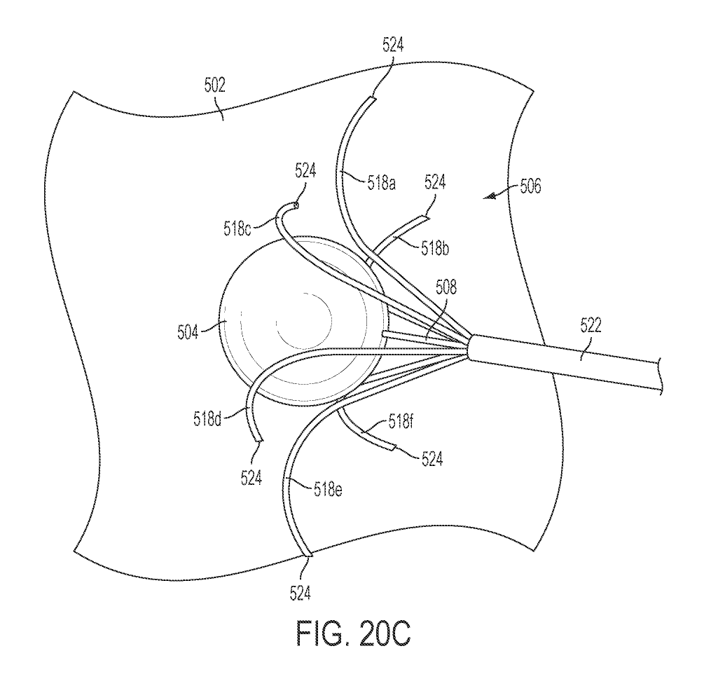

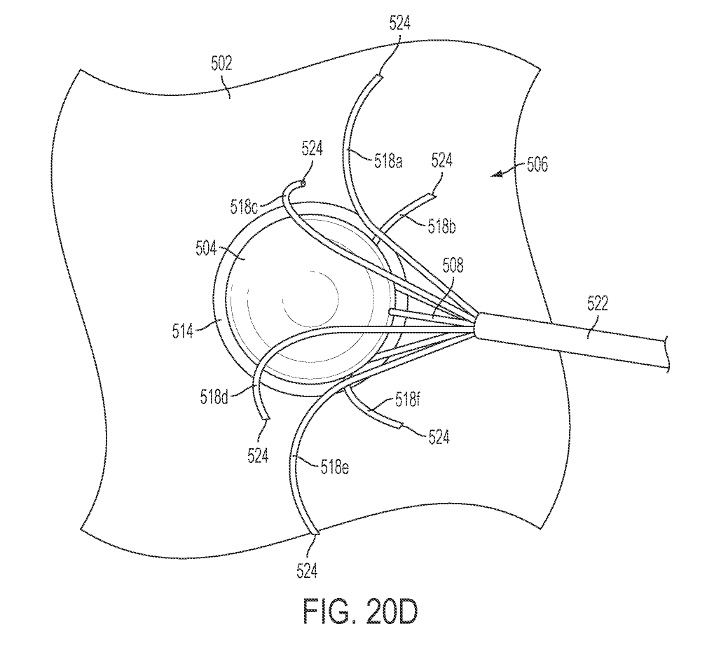

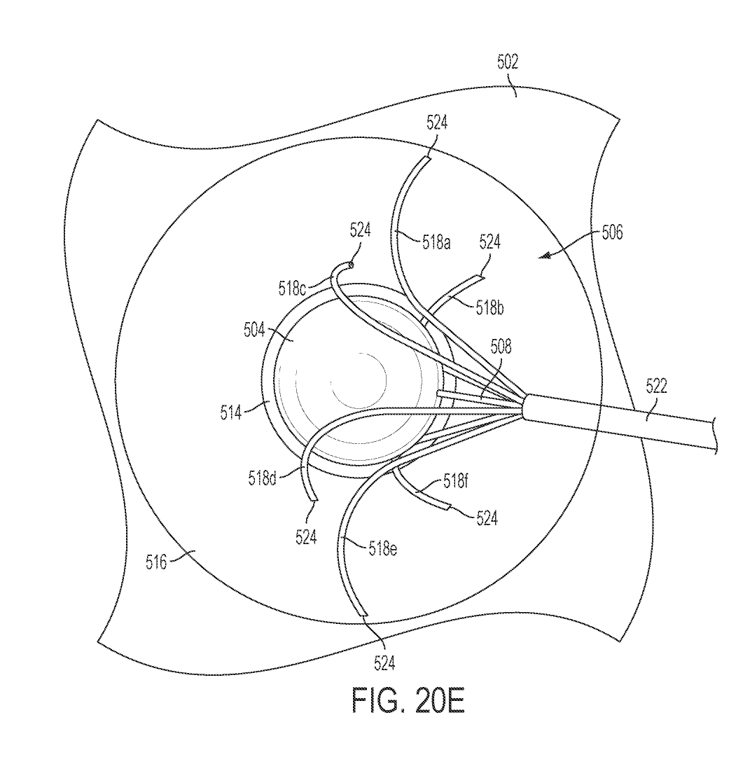

FIGS. 20A-E illustrate one implementation of a method of debulking a tumor and causing a specific systemic response by employing electroporation techniques. FIG. 20A shows a tumor 504 embedded inside the liver 502. FIG. 20B shows a catheter 522 inserted into the liver 502 and advanced to a position proximate to the tumor 504 to treat the tumor 504. Once the catheter 522 is located proximate to the tumor 504, the probe 506 is advanced through an inner lumen of the catheter 522. As previously discussed, the probe 506 comprises a central electrode 508. FIG. 20C shows a distal end of the central electrode 508 inserted into the tumor 504 and a plurality of injection needles 518ₐ-518_{f} surrounding the tumor 504. A proximal end of the electrode 508 (not shown) and a second, return, electrode are configured to couple to corresponding electrical conductors 18a,b, handpiece 16, activation switch 62, and energy source 14 (as described herein with reference to FIG. 1). A proximal end of the injection needles 518ₐ-518_{f} is coupled to a fluid source 524. A distal end of the injection needles 518ₐ-518_{f} comprises a needle 524 for injecting fluids into tissue such as the liver 502. The needle 524 is formed like an ordinary having hypodermic needle having a hollow needle commonly used with a syringe to inject substances into the body or extract liquids from the body. After inserting the electrode 508 into the tumor 504, a fluid is injected into the liver 502 tissue surrounding the tumor 504 using the injection needles 518ₐ-518_{f}. As previously discussed, in one embodiment, a DNA plasmid is injected into the liver 502 tissue with the injection needles 518ₐ-518_{f} before electrode 508 is energized with pulses from the energy source 14. FIG. 20D shows the formation of a necrotic zone, i.e., necrotic Zone A, created by a combination of electrical pulses delivered by the central electrode 508 and injection of DNA plasmid 520 with the injection needles 518ₐ-518_{f}. The volume of Zone A is defined by perimeter 514 and envelops the tumor 504. The perimeter 514 marks the boundary where the electric field strength E_{A} (V/cm) is above the necrotic threshold of about 700 V/cm. FIG. 20E shows the formation of a reversible zone, i.e., Zone B, created by energizing the electrode 508. The volume of Zone B is defined by perimeter 516. Zone B is located further away from the center 510 of the central electrode 508 than Zone A. Zone B surrounds Zone A and the perimeter 516 between Zone a and Zone B marks the boundary where the electric field strength E_{B} (V/cm) is well below the necrotic threshold but still strong enough to cause poration of the plasma membranes.

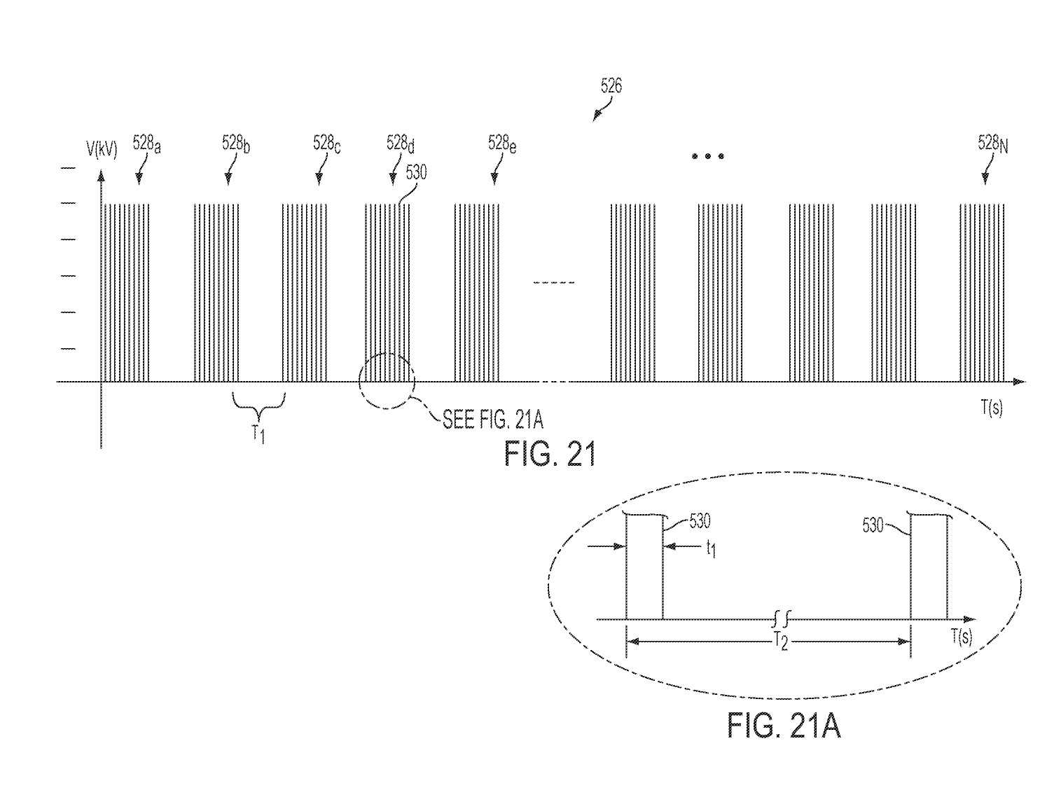

FIG. 21 is a graphical illustration of a sequence of electrical pulses 526 (e.g., a pulse train) produced by the energy source 14 (as described herein with reference to FIG. 1) which can be applied to the central electrode 508 for the treatment or debulking of a tumor. Voltage (V) in kV is shown along the vertical axis and time (T) in seconds is shown along the horizontal axis. With reference now to FIGS. 20A-E and 21, in one embodiment, the pulse train 526 can be applied to the electrode 508 to create the necrotic Zone A and the reversible Zone B for treating or debulking the tumor 504 growing in the liver 502. In one embodiment, the pulse train 526 comprises a sequence of electrical pulse bursts 528ₐ-528_{N}, where N is a positive integer. Each of the pulse bursts 528ₐ-528_{N} having a period Tₒ (e.g., fₒ), which includes an “on” period and an “off” period and includes a plurality of individual pulses 530. In the illustrated embodiment, the “on” and “off” period T₁ is about 1s in duration (e.g., a frequency f₁ of about ½ Hz). FIG. 21A shows a magnified portion of the individuals pulses 530 that compose each of the pulse bursts 528ₐ-528_{N}, and more particularly, the pulse burst 528_{d}. Each of the pulses 530 has a pulse width t₁ and repeat at a period of T₂ (e.g., f₂). In the illustrated embodiment, the pulse width t₁ is about 2 μs and T₂ is about 5 ms (e.g., a frequency f₂ of about 200 Hz). In the illustrated embodiment, the amplitude of the pulses 530 is about 3 kV.

Liver malignancies are growing worldwide. Conventional treatment alternatives for hepatic liver malignancies such as hepatocellular carcinoma (HCC) and colorectal liver metastases (CRLM) include percutaneous ethanol ablation (PEI), transcatheter embolization (TACE), and ablation. Ablation is performed as an open procedure, laparoscopically, and percutaneously. Patients with HCC are often not candidates for resection due to the underlying disease, while 75% of CRLM are not resectable. Due in part to the difficulty of accessing the liver percutaneously, the recurrence rate after ablation has been reported to be about 3.5% and about 26.4% (p<0.0001) for surgical and percutaneous procedures respectively. Yet the rate of morbidity has been shown to be 15.3% v. 2.4%, (p=0.044) for surgical and percutaneous procedures respectively. It may be possible to increase effectiveness and reduce morbidity by treating liver malignancies using NOTES™ and percutaneous procedures.

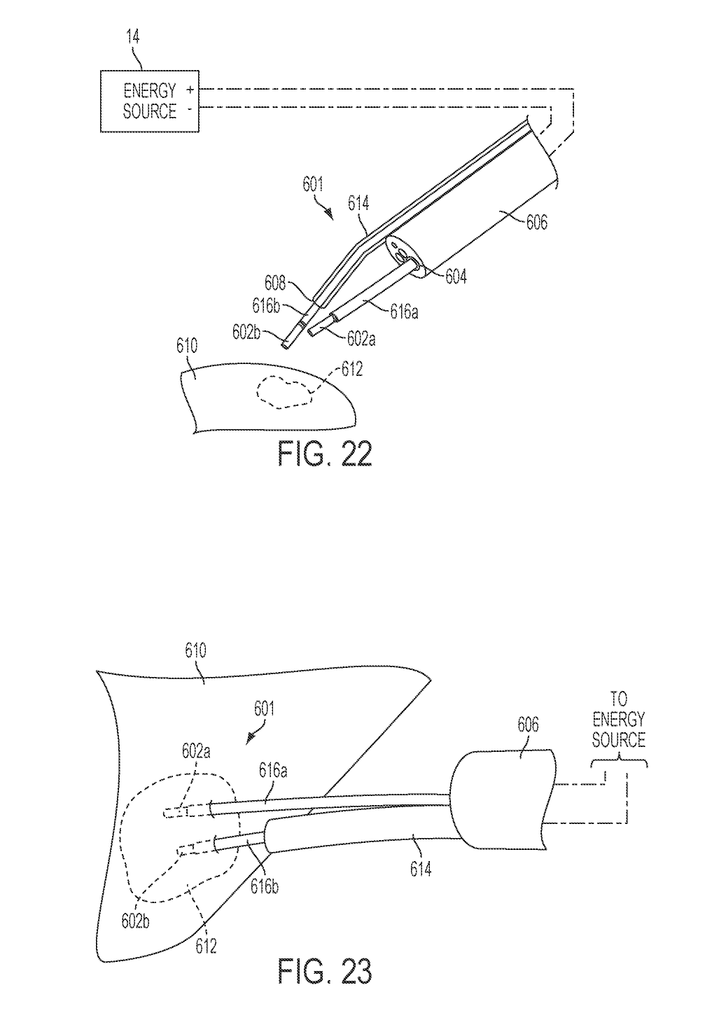

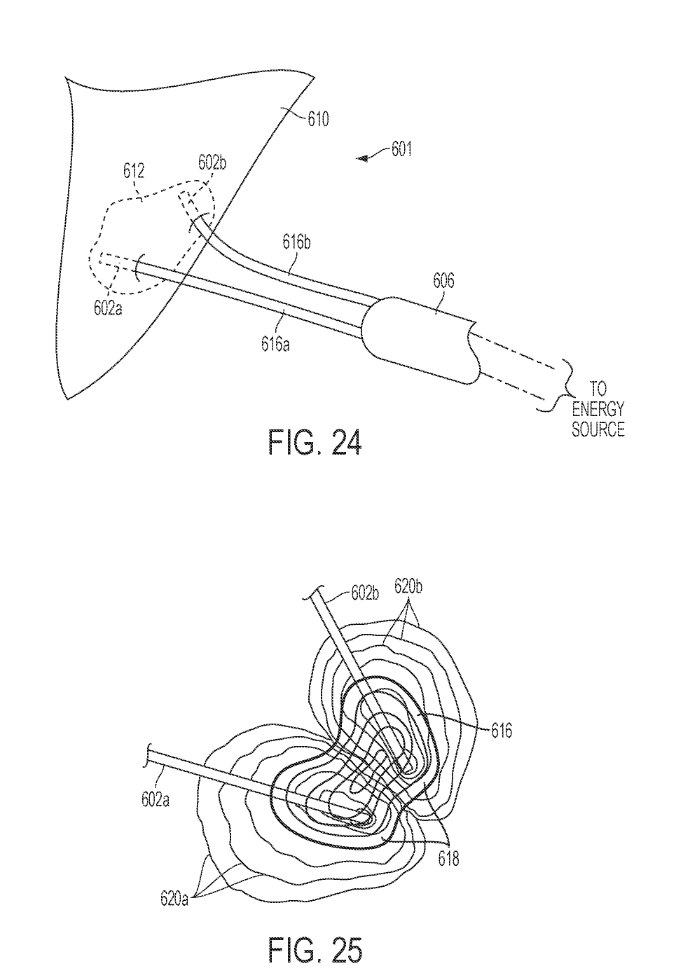

FIGS. 22-24 illustrate one embodiment of an ablation device 601 comprising a center inner electrode 602a and an outer electrode 602b, which are non-parallel relative to each other. The inner center electrode 602a is movably disposed in a channel 604 of a gastroscope 606 and the outer electrode 602b is located outside the gastroscope 606 through a channel 608 of a manipulation device 614, for example. On example of a manipulation device 614 is a device made by Ethicon Endosurgery, Inc. and commonly referred to as an Ardvark device. In one embodiment, the manipulation device 614 attaches to the outside of a flexible scope, such as, for example, the gastroscope 606. A hollow channel can be slid down the outside of an external track and advanced beyond the distal end of the scope. The channel can be articulated independent of the scope. Therefore an electrode that is advanced through the channel can be articulated independent of an electrode advanced through a channel in the scope. The second, outer, electrode 602b is independently operable from the inner electrode 602a. The gastroscope 606 may be advanced through the inner anatomy using conventional natural orifice techniques. For example, the gastroscope 606 may be inserted in the patient's mouth and advanced through the esophagus into the patient's stomach. From the stomach, the gastroscope 606 pierces through the stomach wall and is advanced to the liver 610 through the pierced opening. Once the gastroscope 606 has exited the gastric wall, the center electrode 602a is placed into the center of a tumor 612 growing inside the liver 610. In one example technique, the center electrode 602a may be placed under some type of guidance which may be triangulation (with sensors attached top the electrodes), computed tomography (CT), ultrasonography or other similar placement technique. In one embodiment, the center electrode 602a may have a coil like shape (e.g., corkscrew) which anchors into the tumor 612.

FIG. 25 shows the outer electrode 602b advanced into the tumor 612 while a finite element rendering of a threshold of necrosis 616 showing the electric field isosurface 616 defining the boundary of the necrotic zone 618 is constantly updated and overlaid on a CT image on a display. Contour lines 620a and 620b help to visualize the strength of the electric field around the electrodes 602a,b, respectively. The manipulation device 614 enables the operator to precisely control the placement of the outer electrode 602b. Proximal ends of the electrodes 602a,b are coupled to corresponding electrical conductors 18a,b, handpiece 16, activation switch 62, and energy source 14 (as described herein with reference to FIG. 1). High voltage DC pulses as described herein may be applied to the tumor 612 for ablation treatment. In the illustrated embodiment, the contour lines 620a closest to the distal end of the energized electrode 602a represents electric field strength of about 3 kV/cm with the strength of the electric field decreasing to about 1.5 kV/cm near the outer contour lines 620a. The contour lines 620b closest to the distal end of the return or ground electrode 602b represent electric field strength of 1.5 kV/cm and decreases substantially near the outer contour lines 620b.

In one embodiment, treatment of the tumor 612 may be effected by applying a sequence of electrical pulses to the inner electrode 602a and applying a ground potential to the outer electrode 602b. It will be appreciated that, in one embodiment, the polarity may be reversed such that the sequence of electrical pulses is applied to the outer electrode 602b and the ground potential is applied to the inner electrode 602a. It can be further appreciated that, in one embodiment, the sequence of electrical pulses can be applied differentially between (1) the inner electrode 602a and (2) the outer electrode 602b. In one embodiment, the sequence of pulses have amplitudes in the range of about ±100 to about ±10,000VDC, pulse lengths (e.g., pulse width, pulse duration) in the range of about 1 μs to about 100 ms, and frequencies in the range of about 1 Hz to about 10,000 Hz.

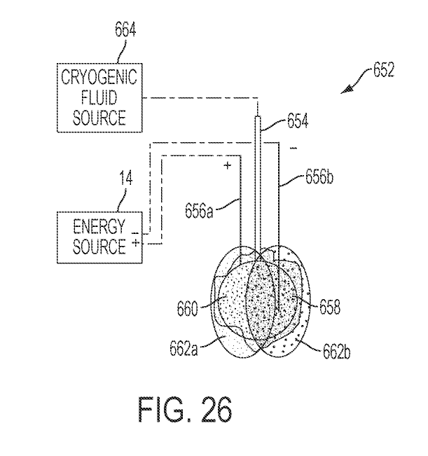

FIG. 26 is a diagram illustrating a combined cryogenic and irreversible electroporation (IRE) treatment of a tumor. Cryoablation has been used for many years as a way to cause tumor necrosis. Likewise, electroporation (both reversible and irreversible) have been used to cause tissue necrosis. The mechanism of cell lysis is similar for each. It will be appreciated that lysis refers to the death of a cell by breaking of the cellular membrane, causing the contents to spill out and compromising its integrity. In one embodiment, a combined cryogenic and IRE probe 652 comprises three elements, all of which could be incorporated into a single catheter. In the illustrated embodiment, the probe 652 comprises a cryo-probe 654 and two IRE electrodes 656a, 656b. A proximal end of the cryo-probe 654 is coupled to a source of cryogenic fluid 664. The electrodes 656a,b are coupled to corresponding electrical conductors 18a,b, handpiece 16, activation switch 62, and energy source 14 (as described herein with reference to FIG. 1). In one embodiment, a proximal end of one IRE electrode 656a is coupled to a positive (+) output of the energy source 14 (as described herein with reference to FIG. 1) and a proximal end of the other IRE electrode 656b is coupled to the negative (−) output of the energy source 14.

The probe 652 is advanced to a tumor 658 site via a catheter or other tube that can be inserted into a body cavity, duct or vessel to provide access by surgical instruments to a tissue treatment site. Once the catheter and the probe 652 are located proximate to the tumor 658, distal ends of the cryo-probe 654 and the IRE electrode 656a,b are advanced from the catheter and located proximate to the tumor 658.

Once properly positioned proximate the tumor 658, treatment of the tumor 658 can be effected by cryogenically cooling the cryo-probe 654 with cryogenic fluid to form a cryogenic zone 660. The cryogenic zone 660 is in the form of an ice ball that forms around the distal end of the cryo-probe 654. The cryogenic zone 660 is generally symmetrically formed around the cryo-probe 654. Once the cryogenic zone is formed, a sequence of electrical pulse can be applied to the tumor 658 by energizing the IRE electrodes 656a,b. This creates IRE zones 662a, 662b that take the shape of two lobes as the electrodes 656a,b are moved further apart. The combined cryogenic zone 658 and IRE zones 662a,b yield a larger kill zone for treating the tumor 658. Furthermore, the damage inflicted by cryogenically freezing the tumor 658 tissue could damage the individual cells and possibly lower the required electric field threshold of necrosis. In one embodiment, the sequence of electrical pulses is applied to the first electrode 662a and ground potential is applied to a second electrode 662b. It will be appreciated that, in one embodiment, the polarity may be reversed such that the sequence of electrical pulses is applied to the second electrode and the ground potential is applied to the first electrode 662a. It can be further appreciated that, in one embodiment, the sequence of electrical pulses can be applied differentially between (1) the first electrode 662a and (2) the second electrode 662b. In one embodiment, the sequence of pulses have amplitudes in the range of about ±100 to about ±10,000VDC, pulse lengths (e.g., pulse width, pulse duration) in the range of about 1 μs to about 100 ms, and frequencies in the range of about 1 Hz to about 10,000 Hz.

Electric field strength (kV/cm) is shown along the vertical axis and pulse width (sec) is shown along the horizontal axis. The value of electric field strength (and greater) that will cause cell death for a given value of pulse width can be determined based on the necrotic threshold curve 702. Likewise, the value of pulse width (and longer) that will cause cell death for a given value of electric field strength can be determined based on the curve 702. Although the curve 702 was produced based on empirical measurements, it has a theoretical basis. For example, the pulse width will determine whether a cell membrane will charge to a sufficiently high level to cause cell damage and subsequent death. Likewise a shorter pulse width will charge the membranes of the cell organelles and cause damage which will produce the apoptotic cascade to begin. A pulse width on the order of about 100 nsec will cause both to occur.

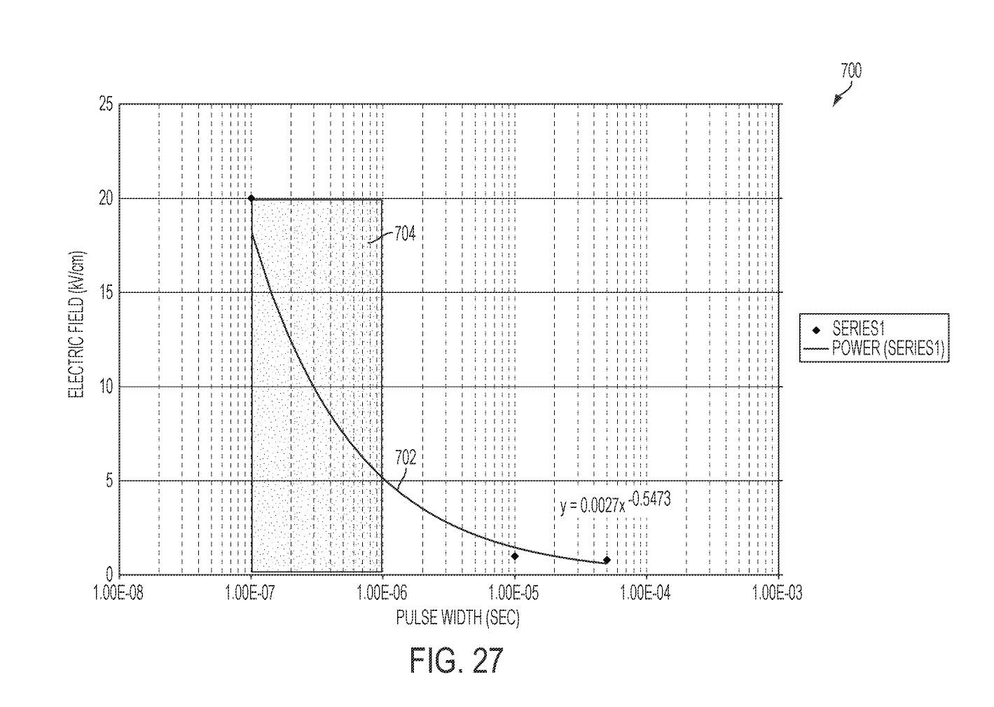

As shown in FIG. 27, a shaded region 704 represents a zone in which both necrosis and apoptosis occur. There are additional advantages to operating in the shaded region 704 over similar devices. One advantage is the reduction of the intensity of muscular contractions when unipolar pulses are applied in-vivo. Excitation of skeletal muscle occurs when nerve impulses travel along mylenated nerve fibers originating in the spinal cord. The action potentials of nerves have a pulse duration of about 0.2 μs. It can be shown that a pulse duration of about 100 nsec or less does not cause an abdominal muscle contraction, whereas pulses with a duration of about 10 μs can cause significant muscle contractions. A pulse width greater than about 100 ns may cause less intense contractions.

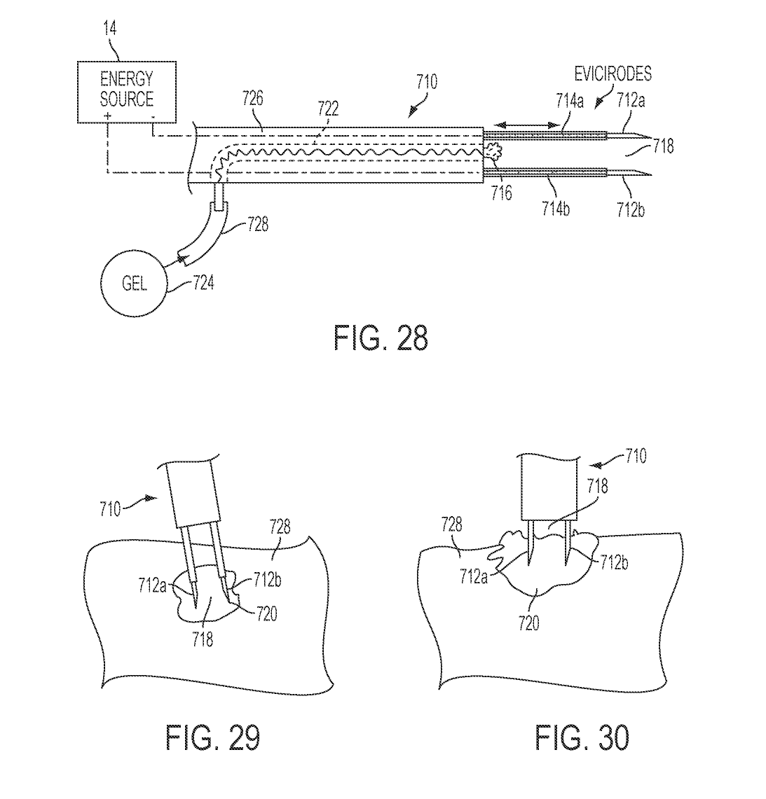

In one embodiment, a unipolar pulse having a pulse duration of 100 ns to about 900 ns can be delivered to a tissue treatment site to cause necrotic death of undesirable tissue cells. Unipolar pulses have been shown to cause cell necrosis by immediately destroying the cell plasma membrane as well as triggering cell apoptosis. There are advantages to both of these mechanisms of cell death. Causing immediate cell death is preferred if the cell is malignant. Nevertheless, cell apoptosis is a more natural death for a cell and therefore more compatible with the natural method with which the immune system “cleans up” the dead cells. FIGS. 28 and 29 illustrate embodiments of devices which cause necrotic cell death using unipolar pulses having nominal pulse duration of about 250 ns.

FIG. 28 illustrates one embodiment of a probe 710 comprising electrodes 712a, 712b, which can deliver unipolar pulses of about 250 ns to a tissue treatment site to cause necrotic cell death. The distal ends of the electrodes 712a,b are exposed metal, e.g., un-insulated. The remaining portion of the electrodes 712a,b includes electrically insulative portion 714a, 714b. A housing 726 supports the electrodes 712a,b. Proximal ends of the electrodes 712a,b are coupled to corresponding electrical conductors 18a,b, handpiece 16, activation switch 62, and energy source 14 (as described herein with reference to FIG. 1). According to the curve 702 shown in FIG. 27, when the pulse width is less than 1 μs, the electric field strength required to cause cell necrosis increases significantly. To produce a reasonable zone of necrosis, a very high voltage would be required (>10 kVDC). Such a high voltage, however, will increase the chance that a breakdown in air or an arc will form in the space 718 between the un-insulated conductive portions of the electrodes 712a,b are not fully embedded in the tissue 728 as shown in FIG. 29. On the other hand, an arc can occur, for example, when the un-insulated conductive portions of the two electrodes 712a,b are not fully embedded into the tissue 728 and leave the space 718 exposed as shown in FIG. 30, where the exposed space 718 provides an air gap in an arc form at very high voltages (>10 kVDC). As shown in FIG. 28, to prevent arcing in the space 718 between the un-insulated conductive portions of the two electrodes 712a,b, in one embodiment, a channel 722 is formed or provided within the housing 726 portion of the probe 710 to supply a gel 716 from a gel source 724 to the distal end of the probe 710. The channel 722 may be formed integrally within the housing 726 or may be a separate tube or lumen. If the tumor 720 is at the surface of an organ 728 as shown in FIG. 30, it is possible that the exposed electrodes 712a,b will be exposed and create an arc in the space 718 between the electrodes 712a,b. In this case, the gel 716 can be continuously supplied to the space 718 to displace the air in the space 718 and prevent an arc from forming. The gel 718 may be any water-based, water-soluble lubricant such as KY® Jelly produced by Johnson & Johnson.

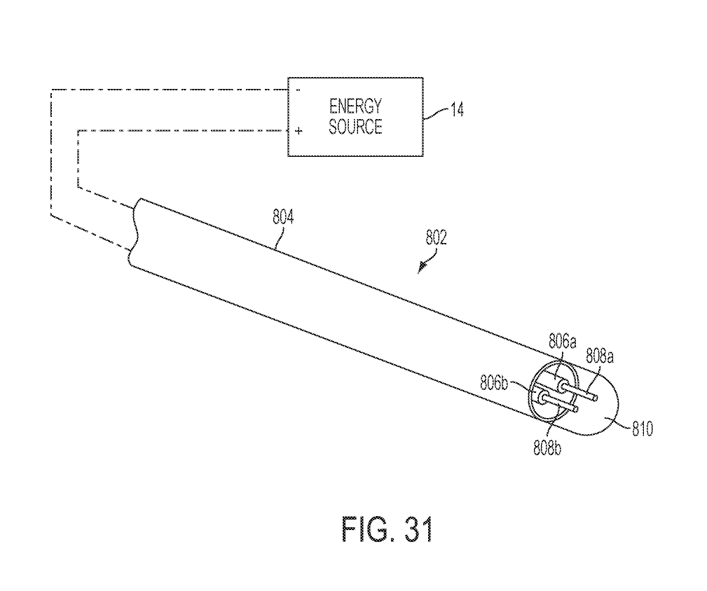

FIG. 31 illustrates one embodiment of a device 802 for producing an acoustic wave using high voltage discharge. As discussed throughout this specification, high voltage (about 3 kV) DC pulses of short duration (about 10 μs) can be used to cause cell necrosis in-vivo. However, during a pulse discharge in the tissue, a sound (e.g., an acoustic wave) can be heard. The sound is most likely due to the rapidly changing voltage at the electrode surface. The following embodiment is generally directed to a device that produces such an acoustic wave for the purpose of treatment. One possible application of such an acoustic devise is for producing an acoustic wave strong enough to break stones in the kidney, for example. The device 802 comprises a flexible catheter 804, which contains two electrical conductors 806a, 806b. The electrical conductors 806a,b have insulation removed at the distal end to expose the electrically conductive portions of first and second electrodes 808a, 808b. Proximal ends of the electrodes 808a,b are coupled to corresponding electrical conductors 18a,b, handpiece 16, activation switch 62, and energy source 14 (as described herein with reference to FIG. 1).

The exposed electrical conductors 806a,b are embedded in a resilient, pliable material such as, for example, silicone. The material forms a pliable dome-like structure 810 over the electrically conductive portions 808a, 808b. The pliable dome-like structure 810 acts as an electrical load when high voltage short duration pulses (about 3 kV at about 10 μs pulse duration) are applied to the electrical conductors 806a,b by the energy source 14 (as described herein with reference to FIG. 1). When a pulse is applied to the electrical conductors 806a,b an acoustic wave is produced in the pliable dome-like structure 810.

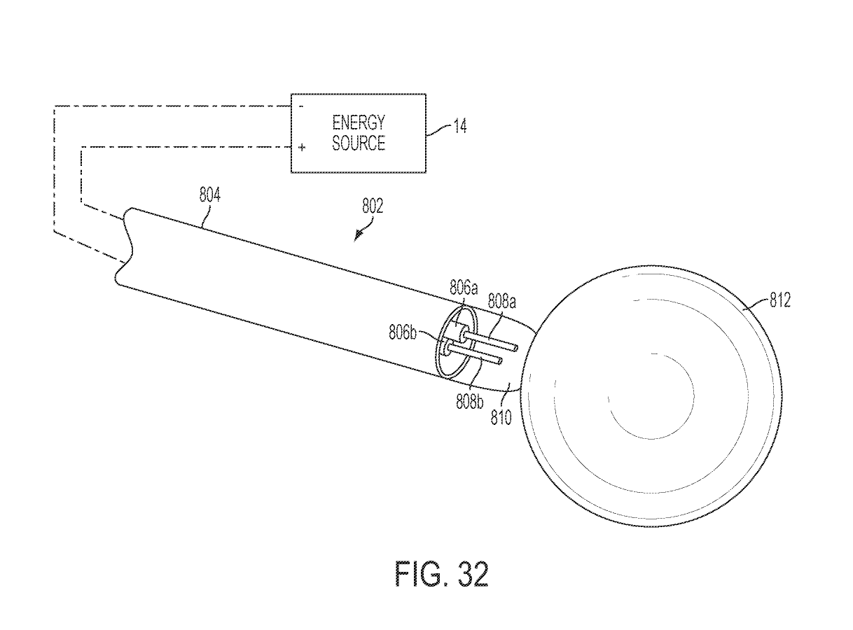

FIG. 32 shows the device 802 with the dome-like structure 810 in contact with a stone 812. As high voltage short repetition pulses are applied to the electrical conductors 806a,b, the acoustic wave produced in the dome-like structure 810 is transferred to the stone 812. Repeated pulses can be applied until the stone 812 is fractured.

The embodiments of the electrical ablation devices described herein may be introduced inside a patient using minimally invasive or open surgical techniques. In some instances it may be advantageous to introduce the electrical ablation devices inside the patient using a combination of minimally invasive and open surgical techniques. Minimally invasive techniques provide more accurate and effective access to the treatment region for diagnostic and treatment procedures. To reach internal treatment regions within the patient, the electrical ablation devices described herein may be inserted through natural openings of the body such as the mouth, anus, and/or vagina, for example. Minimally invasive procedures performed by the introduction of various medical devices into the patient through a natural opening of the patient are known in the art as NOTES™ procedures. Surgical devices, such as an electrical ablation devices, may be introduced to the treatment region through the working channels of the endoscope to perform key surgical activities (KSA), including, for example, electrical ablation of tissues using irreversible electroporation energy. Some portions of the electrical ablation devices may be introduced to the tissue treatment region percutaneously or through small—keyhole—incisions.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, anus, and/or vagina). A rigid endoscope may be introduced via trocar through a relatively small—keyhole—incision incisions (usually 0.5-1.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

Once an electrical ablation device is inserted in the human body internal organs may be reached using trans-organ or translumenal surgical procedures. The electrical ablation device may be advanced to the treatment site using endoscopic translumenal access techniques to perforate a lumen, and then, advance the electrical ablation device and the endoscope into the peritoneal cavity. Translumenal access procedures for perforating a lumen wall, inserting, and advancing an endoscope therethrough, and pneumoperitoneum devices for insufflating the peritoneal cavity and closing or suturing the perforated lumen wall are well known. During a translumenal access procedure, a puncture must be formed in the stomach wall or in the gastrointestinal tract to access the peritoneal cavity. One device often used to form such a puncture is a needle knife which is inserted through the working channel of the endoscope, and which utilizes energy to penetrate through the tissue. A guidewire is then feed through the endoscope and is passed through the puncture in the stomach wall and into the peritoneal cavity. The needle knife is removed, leaving the guidewire as a placeholder. A balloon catheter is then passed over the guidewire and through the working channel of the endoscope to position the balloon within the opening in the stomach wall. The balloon can then be inflated to increase the size of the opening, thereby enabling the endoscope to push against the rear of the balloon and to be feed through the opening and into the peritoneal cavity. Once the endoscope is positioned within the peritoneal cavity, numerous procedures can be performed through the working channel of the endoscope.

The endoscope may be connected to a video camera (single chip or multiple chips) and may be attached to a fiber-optic cable system connected to a “cold” light source (halogen or xenon), to illuminate the operative field. The video camera provides a direct line-of-sight view of the treatment region. The abdomen is usually insufflated with carbon dioxide (CO₂) gas to create a working and viewing space. The abdomen is essentially blown up like a balloon (insufflated), elevating the abdominal wall above the internal organs like a dome. CO₂ gas is used because it is common to the human body and can be removed by the respiratory system if it is absorbed through tissue.

Once the electrical ablation devices are located at the target site, the diseased tissue may be electrically ablated or destroyed using the various embodiments of electrodes discussed herein. The placement and location of the electrodes can be important for effective and efficient electrical ablation therapy. For example, the electrodes may be positioned proximal to a treatment region (e.g., target site or worksite) either endoscopically or transcutaneously (percutaneously). In some implementations, it may be necessary to introduce the electrodes inside the patient using a combination of endoscopic, transcutaneous, and/or open techniques. The electrodes may be introduced to the tissue treatment region through a working channel of the endoscope, an overtube, or a trocar and, in some implementations, may be introduced through percutaneously or through small—keyhole—incisions.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. An electrical ablation apparatus, comprising:
an injector catheter electrode having a proximal end configured to couple to an energy source and a fluid source and a distal end defining an injection needle, the injector catheter electrode defining an electrically conductive hollow channel for communicating a fluid from the fluid source to a treatment site; and
a balloon electrode in fluid communication with a balloon catheter, the balloon catheter having a proximal end configured to couple to the energy source and the fluid source and a distal end configured to inflate the balloon electrode.

2. The electrical ablation apparatus of claim 1, comprising:
an energy source coupled to the proximal end of the injector catheter electrode and the balloon electrode, wherein the energy source is configured to deliver a sequence of electrical pulses having amplitudes in the range of about ±100 to about ±10,000VDC, pulse lengths in the range of about 1 μs to about 100 ms, and frequencies in the range of about 1 Hz to about 10,000 Hz; and
a fluid source coupled to the proximal end injector catheter electrode and the balloon electrode.

3. A method of treating tissue, comprising:
obtaining the apparatus of claim 2;
advancing the injector catheter electrode and the balloon electrode to a tissue treatment site with an endoscope;
injecting an electrically conductive fluid proximal to the treatment site with the injector catheter electrode;
forming a bleb filled with the electrically conductive fluid;
inflating the balloon electrode with an electrically conductive fluid; and
applying a sequence of electrical pulses to the injector catheter electrode, wherein the sequence of electrical pulses have amplitudes in the range of about ±100 to about ±10,000VDC, pulse lengths in the range of about 1 μs to about 100 ms, and frequencies in the range of about 1 Hz to about 10,000 Hz; and
applying a ground potential to the balloon electrode.

4-11. (canceled)

12. An electro-chemotherapy apparatus, comprising:
an electrode having a proximal end configured to electrically couple to an energy source and a distal end configured for effecting treatment of a tissue mass, wherein the first electrode is deployable to a tissue treatment region through a catheter; and
at least one injection needle having a proximal end configured to fluidically couple to a fluid source and a distal end configured to inject fluid into the tissue treatment region, wherein the at least one injection needle is deployable to the tissue treatment region through the catheter.

13. The electro-chemotherapy apparatus of claim 12, comprising:
an energy source electrically coupled to the electrode, wherein the energy source is configured to deliver a sequence of electrical pulses having amplitudes in the range of about ±100 to about ±10,000VDC, pulse lengths in the range of about 1 μs to about 100 ms, and frequencies in the range of about 1 Hz to about 10,000 Hz; and
a fluid source fluidically coupled to the at least one injection needle, wherein the fluid source comprises a DNA plasmid.

14. The electro-chemotherapy apparatus of claim 12, comprising:
a plurality of injection needles having proximal ends configured to fluidically couple to the fluid source and distal ends configured to inject fluid into the tissue treatment region, wherein the plurality of injection needles are deployable to the tissue treatment region through the catheter.

15. An electro-chemotherapy method, comprising:
obtaining the apparatus of claim 14;
advancing the electrode through the catheter;
inserting the electrode into the tissue treatment region;
advancing the at least one injection needle through the catheter;
injecting a DNA plasmid into the tissue treatment region; and
applying a sequence of electrical pulses to the electrode, the sequence of electrical pulses having amplitudes in the range of about ±100 to about ±10,000VDC, pulse lengths in the range of about 1 μs to about 100 ms, and frequencies in the range of about 1 Hz to about 10,000 Hz.

16. The electro-chemotherapy method of claim 15, comprising:
forming a necrotic zone by an electric filed that is greater than about 700V/cm; and
forming a reversible poration zone by an electric field that is less than about 700V/cm.

17. The electro-chemotherapy method of claim 15, comprising:
applying a sequence of electrical pulse bursts to the electrode for a period of about one second and turning off the electrical pulse bursts for a period of about one second, wherein the electrical pulse burst comprises a plurality of pulses each having a duration of about 2 μs and a frequency of about 200 Hz.

18. An electrical ablation apparatus, comprising:
a first electrode; and
a second electrode coupled to a manipulation device for controlling the placement of the outer electrode;
wherein the first electrode and the second electrode are non-parallel relative to each other and the outer electrode is independently operable from the first electrode; and
wherein the first electrode is locatable within the tissue treatment region using a guidance system selected from one of a triangulation system, computed tomography (CT), and ultrasonography.

19. The electrical ablation apparatus of claim 18, comprising:
a display for showing a boundary of cellular necrosis overlaid on a CT image for guiding the placement of the second electrode during the ablation process.

20. The electrical ablation apparatus of claim 18, comprising:
an energy source electrically coupled to the first and second electrodes, wherein the energy source is configured to deliver a sequence of electrical pulses having amplitudes in the range of about ±100 to about ±10,000VDC, pulse lengths in the range of about 1 μs to about 100 ms, and frequencies in the range of about 1 Hz to about 10,000 Hz.

21-34. (canceled)

35. An electrical ablation apparatus, comprising:
first and second electrodes having a plate-like shape and a threaded opening;
a third electrode having first and second threaded ends configured to threadably engage the first and second threaded openings formed in the respective first and second electrodes, wherein the third electrode comprises a conductive portion between the first and second threaded ends and electrically insulative portions between the conductive portion and the first and second threaded ends, and wherein the conductive portion is electrically isolated from the first and second electrodes.

36. The electrical ablation apparatus of claim 35, comprising:
an energy source coupled to the first, second, and third electrodes, wherein the energy source configured to deliver a sequence of electrical pulses having amplitudes in the range of about ±100 to about ±10,000VDC, pulse lengths in the range of about 1 μs to about 100 ms, and frequencies in the range of about 1 Hz to about 10,000 Hz.

37. A method of treating tissue, comprising:
obtaining the apparatus of claim 36;
inserting the third electrode through a tumor embedded in a mass of tissue;
threadably engaging the first and second electrodes to respective first and second end of the third electrode;
applying a sequence of electrical pulses to the third electrode, wherein the sequence of electrical pulses have amplitudes in the range of about ±100 to about ±10,000VDC, pulse lengths in the range of about 1 μs to about 100 ms, and frequencies in the range of about 1 Hz to about 10,000 Hz; and
applying a ground potential to the first and second electrodes.

38. The method of claim 37, comprising:
compressing the mass of tissue by rotating the third electrode and threadably engaging the first and second electrodes to cause the first and second electrodes to advance toward each other; and
repeating the application of the sequence of electrical pulses.

39. A method of treating the prostate, comprising:
inserting a catheter electrode into a lumen defined by the urethra;
advancing the catheter electrode to a location proximate to the prostate;
puncturing an opening through a wall of the urethra;
advancing the catheter electrode into the prostrate through the opening formed in the wall of the urethra;
inserting a balloon electrode into the anus;
advancing the balloon electrode into the rectum to a location proximate the prostate;
inflating the balloon electrode with an electrically conductive fluid; and
applying a sequence of electrical pulses to the catheter electrode, the sequence of electrical pulses having amplitudes in the range of about ±100 to about ±10,000VDC, pulse lengths in the range of about 1 μs to about 100 ms, and frequencies in the range of about 1 Hz to about 10,000 Hz; and
applying a ground potential to the balloon electrode.

40. A method of treating hepatic tumors, comprising:
inserting a first electrode into a hepatic tumor;
inserting a second electrode into the hepatic artery;
advancing the second electrode to arterial branches supplying blood to the hepatic tumor;
applying a sequence of electrical pulses to the first electrode, the sequence of electrical pulses having amplitudes in the range of about ±100 to about ±10,000VDC, pulse lengths in the range of about 1 μs to about 100 ms, and frequencies in the range of about 1 Hz to about 10,000 Hz; and
applying a ground potential to the second electrode.

41. The method of claim 40, comprising:
injecting a conductive fluid into the arterial system proximate the second electrode; and
re-applying the sequence of electrical pulses to the first electrode.

42. The method of claim 40, comprising:
administering a chemical agent through the hepatic artery; and
re-applying the sequence of electrical pulses to the first electrode.

43. An ablation apparatus, comprising:
first and second electrodes coupled to an energy source, wherein the energy source electrically coupled to the first and second electrodes, wherein the energy source is configured to deliver a sequence of electrical pulses having amplitudes in the range of about ±100 to about ±10,000VDC, pulse lengths in the range of about 1 μs to about 100 ms, and frequencies in the range of about 1 Hz to about 10,000 Hz; and
a cryogenic probe coupled to a cryogenic fluid source;
wherein the cryogenic probe is configured to create a cryogenic zone in the tissue treatment region prior to a sequence of electrical pulses being applied to the first and second electrodes.

44. A method of treating tissue, comprising:
obtaining the apparatus of claim 43;
delivering a cryogenic fluid to the tissue treatment region with the cryogenic probe;
applying a sequence of electrical pulses to the first electrode, the sequence of electrical pulses having amplitudes in the range of about ±100 to about ±10,000VDC, pulse lengths in the range of about 1 μs to about 100 ms, and frequencies in the range of about 1 Hz to about 10,000 Hz; and
applying a ground potential to the second electrode.

45. An ablation apparatus, comprising:
first and second electrodes coupled to an energy source, wherein the energy source electrically coupled to the first and second electrodes, wherein the energy source is configured to deliver a sequence of electrical pulses having amplitudes in the range of about ±100 to about ±10,000VDC, pulse lengths in the range of about 1 μs to about 100 ms, and frequencies in the range of about 1 Hz to about 10,000 Hz;
a housing for supporting the first and second electrodes; and
a channel located within the housing fluidically coupled to a source of gel for delivering the gel to a distal portion of the housing to a space between the first and second electrodes.

46. A method of treating tissue, comprising:
obtaining the apparatus of claim 45;
delivering the gel to the distal portion of the housing to the space between the first and second electrodes;
applying a sequence of electrical pulses to the first electrode, the sequence of electrical pulses having amplitudes in the range of about ±100 to about ±10,000VDC, pulse lengths in the range of about 1 μs to about 100 ms, and frequencies in the range of about 1 Hz to about 10,000 Hz; and
applying a ground potential to the second electrode.

47. An apparatus for producing an acoustic wave suitable for treating a stone, the apparatus comprising:
first and second electrodes coupled to an energy source, wherein the energy source electrically coupled to the first and second electrodes, wherein the energy source is configured to deliver a sequence of electrical pulses having amplitudes in the range of about ±100 to about ±10,000VDC, pulse lengths in the range of about 1 μs to about 100 ms, and frequencies in the range of about 1 Hz to about 10,000 Hz;
a housing for supporting the first and second electrodes; and
a resilient dome-like structure formed at a distal end of the first and second electrodes.

48. A method of treating a stone, comprising:
obtaining the apparatus of claim 47;
contacting the dome-like structure with the stone;
producing an acoustic wave by applying a sequence of electrical pulses to the first electrode, the sequence of electrical pulses having amplitudes in the range of about ±100 to about ±10,000VDC, pulse lengths in the range of about 1 μs to about 100 ms, and frequencies in the range of about 1 Hz to about 10,000 Hz; and
applying a ground potential to the second electrode.
